(19) United States
(12) Patent Application Publication
(10) Pub. No.: US 2022/0273732 A1
(43) Pub. Date: Sep. 1, 2022

(54) PROBIOTIC THERAPIES FOR SOCIAL DEFICIT AND STRESS RESPONSE

(71) Applicant: California Institute of Technology, Pasadena, CA (US)

(72) Inventors: Sarkis K MAZMANIAN, Pasadena, CA (US); Wen-Li WU, Pasadena, CA (US); Mark D ADAME, Pasadena, CA (US); Brittany NEEDHAM, Pasadena, CA (US)

(21) Appl. No.: 17/652,464

(22) Filed: Feb. 24, 2022

Related U.S. Application Data
(60) Provisional application No. 63/154,522, filed on Feb. 26, 2021.

(51) Int. Cl. A61K 35/744; A61K 31/567; A61P 25/28; A61B 5/145

(52) U.S. Cl. CPC A61K 35/744; A61K 31/567; A61P 25/28; A61B 5/14546

(57) ABSTRACT

Some embodiments include bacterial species for use in treatment of social behavioral deficit symptoms in a subject in need thereof. The bacterial species can include Enterococcus faecalis. Upon treatment, one or more symptoms of behavioral deficit can be improved in the subject.

### CROSS-REFERENCE TO RELATED APPLICATIONS

The present application claims the benefit of U.S. Provisional Application No. 63/154,522, filed on Feb. 26, 2021, which is hereby incorporated by reference in its entirety.

### STATEMENT REGARDING FEDERALLY SPONSORED R&D

This invention was made with government support under Grant No. MH100556 awarded by the National Institutes for Health. The government has certain rights in the invention.

### REFERENCE TO SEQUENCE LISTING

The present application is being filed along with a Sequence Listing in electronic format. The Sequence Listing is provided as file CALTE158_SEQLIST.TXT created and last modified on Feb. 17, 2022, which is 1,090 bytes in size. The information in the electronic format of the Sequence Listing is incorporated herein by reference in its entirety.

### FIELD

Some embodiments described herein relate generally to probiotic compositions, which can be used to treat social behavioral deficit.

### BACKGROUND

The gut microbiota impacts brain development and function as well as complex behaviors such as anxiety, hyperactivity, vocalization, and social interaction (Sudo, 2004; Clarke, 2013; Neufeld, 2011; Crumeyrolle-Arias, 2014). However, the neural pathways mediating most gut-brain connections remain elusive. Mounting evidence now suggests that interactions between the gut microbiome and the brain are responsible for regulating pathological and normal emotional states. For example, the microbiome is altered in several neuropsychiatric disorders that display social deficits such as ASD, schizophrenia and depression (Kang, 2013; De Palma, 2017; Zheng, 2016), and findings in rodents and humans implicate changes in gut bacteria are a contributing factor in brain morphology, activity, transcriptional patterns, neurogenesis, expression of neurotransmitters, and many complex behaviors (Foster, 2017). Dynamic tuning, quality, and selectivity of social behaviors may also be mediated by the microbiota, and interestingly, several studies have shown that E. faecalis appears to be capable to modulate the nervous system or produce neuroactive metabolites in various paradigms (Kambe, 2020; Mazzoli, 2016; Papasian, 2006; Pessione, 2009; Shiina, 2015; Shimazu, 2004; Takahashi, 2019; Bojovic, 2020). Therefore, discovery of a specific neuronal pathway that responds to signals from the gut may enable interventions to modulate social behaviors through safe, natural, and non-invasive approaches.

### SUMMARY

In accordance with some embodiments described herein, methods for improving a social behavior in a subject in need of such improvement are provided. In some embodiments, the method comprises identifying a subject having a behavioral deficit or a symptom; and administering to the subject a composition comprising an effective amount of one or more Enterococcus bacteria.

Some embodiments provided herein relate to methods of treating a social behavioral deficit in a subject in need thereof. In some embodiments, the methods include identifying a subject having a social behavioral deficit, or a symptom thereof. In some embodiments, the methods include administering to the subject a composition comprising an effective amount of one or more Enterococcus bacteria. In some embodiments, the social behavioral deficit of the subject is improved after administering the composition. In some embodiments, the social behavioral deficit is anxiety, autism spectrum disorder (ASD), schizophrenia, or depression. In some embodiments, a sole active ingredient administered to the subject in the method consists essentially of the one or more Enterococcus bacteria. In some embodiments, the effective amount of the one or more Enterococcus bacteria is in a composition substantially free of bacteria other than the Enterococcus bacteria. In some embodiments, the one or more Enterococcus bacteria comprises E. faecalis. In some embodiments, a sole active ingredient administered to the subject in the method consists essentially of E. faecalis. In some embodiments, the effective amount of the one or more Enterococcus bacteria is in a composition substantially free of bacteria other than the E. faecalis. In some embodiments, an effective amount of one or more Enterococcus bacteria is administered orally. In some embodiments, the behavioral performance is determined by standard behavioral testing. In some embodiments, the composition comprising bacteria within the genus Enterococcus is a probiotic composition. In some embodiments, the composition of bacteria within the genus Enterococcus is a nutraceutical composition. In some embodiments, the composition of bacteria within the genus Enterococcus is a pharmaceutical composition.

Some embodiments provided herein relate to methods of treating a social behavioral deficit in a subject in need thereof. In some embodiments, the methods include identifying a subject having high corticosterone levels or a subject who would benefit from reduced corticosterone levels. In some embodiments, the methods include administering to the subject a composition comprising an effective amount of one or more Enterococcus bacteria to regulate corticosterone levels in the subject. In some embodiments, the methods further include detecting a presence and/or a level of corticosterone. In some embodiments, the corticosterone is expressed differently in normal and behaviorally deficit subjects. In some embodiments, regulating corticosterone levels in the subject comprises inhibition of glucocorticoid signaling. In some embodiments, inhibition of corticosterone signaling in the subject comprises pharmacological or surgical approaches or a combination thereof. In some embodiments, a pharmacological approach includes injection of the corticosterone synthesis blocker, metyrapone. In some embodiments, a surgical approach includes removal of at least one of the two adrenal glands. In some embodiments, the methods further include determining the behavioral performance of the subject prior to and after adjusting the corticosterone level in the subject.

Some embodiments provided herein relate to methods of identifying a subject having a behavioral deficit. In some embodiments, the methods including detecting at least one of a presence and/or level of corticosterone or a level of a product of a gene of the subject selected from the group consisting of: Crh1 (or an ortholog thereof), c-Fos (or an ortholog thereof), Nr3c1 (or an ortholog thereof), a human ortholog of any of the listed genes, or a combination of two or more of the listed genes. In some embodiments, detecting a presence and/or a level of (a) and/or (b), wherein (a) and/or (b) are expressed differently in normal and social behaviorally deficit subjects. In some embodiments, c-Fos expression is detected in brain regions associated with stress responses, including the hippocampal dentate gyms (DG), paraventricular nucleus of the hypothalamus (PVN), or BNST or a combination thereof.

Some embodiments provided herein relate to methods of treating a social behavioral deficit in a subject. In some embodiments, the methods include identifying a subject having a social behavioral deficit. In some embodiments, the methods include reducing corticosterone levels in the subject. In some embodiments, the methods reduce the social behavioral deficit in the subject. In some embodiments, reducing corticosterone levels in the subject includes inhibiting corticosterone synthesis in the subject. In some embodiments, inhibiting corticosterone synthesis in the subject includes pharmacological or surgical approaches or a combination thereof In some embodiments, a pharmacological approach comprises injection of the corticosterone synthesis blocker, metyrapone in the subject. In some embodiments, a surgical approach comprises removal of at least one of the two adrenal glands in the subject. In some embodiments, the methods further include determining the corticosterone levels in the subject prior to and after inhibiting corticosterone synthesis in the subject. In some embodiments, the methods further include determining the social behavior of the subject prior to and after inhibiting corticosterone synthesis in the subject. In some embodiments, reducing corticosterone levels in the subject includes removing an adrenal gland in the subject, antagonizing a glucocorticoid receptor in the subject, inhibiting corticosterone synthesis in the subject, or promoting gut microbiome levels in the subject, or a combination thereof. In some embodiments, removing an adrenal gland comprises removal of at least one of the two adrenal glands. In some embodiments, the methods further include determining the corticosterone levels in the subject prior to and after removing an adrenal gland in the subject. In some embodiments, antagonizing a glucocorticoid receptor comprises administering RU-486 to the subject. In some embodiments, the methods further include determining the corticosterone levels in the subject prior to and after antagonizing a glucocorticoid receptor in the subject. In some embodiments, inhibiting corticosterone synthesis in the subject comprises pharmacological or surgical approaches or a combination thereof. In some embodiments, a pharmacological approach includes injection of the corticosterone synthesis blocker, metyrapone in the subject. In some embodiments, a surgical approach comprises removal of at least one of the two adrenal glands in the subject. In some embodiments, the methods further include determining the corticosterone levels in the subject prior to and after inhibiting corticosterone synthesis in the subject. In some embodiments, promoting gut microbiome levels in the subject includes administering to the subject a composition comprising an effective amount of one or more Enterococcus bacteria.

Some embodiments provided herein relate to methods of treating a social behavioral deficit in a subject in need thereof. In some embodiments, the subject suffers from anxiety, autism spectrum disorder (ASD), schizophrenia, depression or a pathological condition with one or more of the symptoms of social behavioral deficit. In some embodiments, the subject is in need of improving social behavior.

In some embodiments, a sole active ingredient administered to the subject in the method consists essentially of the one or more Enterococcus bacteria. In some embodiments, the effective amount of the one or more Enterococcus bacteria is in a composition substantially free of bacteria other than the Enterococcus bacteria. In some embodiments, the one or more Enterococcus bacteria comprises E. faecalis. In some embodiments, a sole active ingredient administered to the subject in the method consists essentially of E. faecalis. In some embodiments, the effective amount of the one or more Enterococcus bacteria is in a composition substantially free of bacteria other than the E. faecalis. In some embodiments, the effective amount of one or more Enterococcus bacteria comprises at least about 10⁷ colony forming units (cfu), for example at least about 10⁷ cfu, at least about 10⁸ cfu, at least about 10⁹ cfu, at least about 10¹⁰ cfu, at least about 10¹¹ cfu, or at least about 10¹² cfu.

In some embodiments, the composition is a probiotic composition, a nutraceutical, a pharmaceutical composition, or a mixture thereof.

In some embodiments, the method includes detecting at least one of the presence or levels of corticosterone, or the levels of a product of a gene selected from a group consisting of Crh1 (or an ortholog thereof), c-Fos (or an ortholog thereof), Nr3c1 (or an ortholog thereof), a human ortholog of any of the listed genes, or a combination of two or more of the listed genes.

In some embodiments, the method for improving a social behavior in a subject in need of such improvement comprises identifying a subject having a behavioral deficit or a symptom thereof; and reducing corticosterone levels in the subject, thereby reducing the social behavioral deficit in the subject.

In some embodiments, the method includes determining the reference level of corticosterone in normal subjects. In some embodiments, the method includes determining the behavioral performance of the subject prior to and after adjusting the level of corticosterone in the subj ect.

In some embodiments, reducing the level of corticosterone in the subject comprises administering to the subject a composition comprising an effective amount of one or more Enterococcus bacteria.

Some embodiments disclosed herein are related to a method for adjusting the level of corticosterone in the subject suffering from social deficit, comprising inhibiting corticosterone synthesis in the subject, removing an adrenal gland in the subject, antagonizing a glucocorticoid receptor in the subject, promoting gut microbiome levels in the subject or a combination thereof. In some embodiments, reducing the level of corticosterone in the subject comprises inhibiting corticosterone synthesis in the subject. In some embodiments, inhibiting corticosterone synthesis in the subject comprises treatment of the subject with a corticosterone synthesis blocker or removing a gland involved in the production of corticosterone.

Some embodiments provided herein relate to methods for reducing the level of corticosterone in the subject suffering from social deficit, comprising blocking the function of glucocorticoid receptor in the subject. In some embodiments, reducing the level of corticosterone in the subject comprises deleting the gene encoding glucocorticoid receptor in the subject. In some embodiments, the method includes determining the behavioral performance of the subject prior to and after blocking the function or deleting the gene glucocorticoid receptor in the subject.

### BRIEF DESCRIPTION OF THE DRAWINGS

The foregoing and other features of the present disclosure will become more fully apparent from the following description, taken in conjunction with the accompanying drawings. Understanding that these drawings depict only some embodiments in accordance with the disclosure and are therefore, not to be considered limiting of its scope, the disclosure will be described with additional specificity and detail through use of the accompanying drawings.

FIGS. 1A-1J depict regulation of social behavior, activation of stress-related brain regions, and serum corticosterone levels in mice by the gut microbiome. FIG. 1A shows a timeline schematic of germ-free (GF) and antibiotic cocktail (ABX) treatment, and social behavior testing in mice. Iso: single housing; RSI: reciprocal social interaction; w: week; h: hour. FIG. 1B shows social activity tested using the RSI paradigm in specific-pathogen-free (SPF) and GF test mice (subject), in the context of SPF and GF novel mice. N=20 SPF, 19 GF mice per group (subject only); Subject versus GF novel mouse: N=8 mice per group (subject only). FIG. 1C, shows treatment of adult SPF mice with ABX, which reduces social behavior in mice. N=26 mice per group (subject only). FIG. 1D, depicts neuronal activity measured by c-Fos staining of brain sections. Top: schematic of brain regions with high c-Fos expression after social interaction. BNST: bed nucleus of stria terminalis; BLA/LA: basolateral amygdala/lateral amygdala; DG: dentate gyms; PVN: paraventricular nucleus of the hypothalamus. Bottom: Representative maximum intensity projection images of c-Fos staining in SPF, GF, vehicle, and ABX after social interaction. Scale bar=200 μm. LV: lateral ventricle; 3V: third ventricle. FIG. 1E-1F, depicts quantification of c-Fos+ cells in various brain regions of SPF, GF, and ABX mice. N=6 SPF, 6 GF, 5 Vehicle, 5 ABX mice per group. FIGS. 1G-1H shows an elevation in serum corticosterone concentrations after social interaction in GF (FIG. 1G) and ABX (FIG. 111) mice, compared to SPF mice. GF: Subject versus SPF novel mouse: N=13 SPF, 18 GF mice per group; Subject versus GF novel mouse: N=8 mice per group (subject only). ABX: N=30 mice per group. FIG. 1I shows social activity tested using the RSI paradigm in SPF, GF, exGF (4 weeks of age colonization) test mice (subject), in the context of SPF novel mice. N=20 SPF, 19 GF, 8 exGF mice per group (subject only). FIG. 1J shows a reduction in serum corticosterone concentrations social interaction in exGF mice. N=13 SPF, 18 GF, 8 exGF mice per group (subject only). Data represent mean±SEM. Data analyzed by two-tailed unpaired t-test (FIG. 1B, FIG. 1C, FIG. 1E-1H) and one-way ANOVA with Bonferroni's multiple comparison post-hoc test (FIG. 1I and FIG. 1J). *p≤0.05; **p≤0.01; ***p≤0.001; ****p≤0.0001; ND: no difference.

FIGS. 2A-2F depict systemic modulation of glucocorticoid signaling alters social behaviors impacted by gut bacteria. FIG. 2A shows a timeline schematic of ABX treatment, drug treatment, social behavior, adrenalectomy (ADX) procedure, subdiaphragmatic vagotomy (SDV) and sample collection. CMC: carboxymethylcellulose (vehicle); Iso: single housing; MET: metyrapone; RSI: reciprocal social interaction; RU-486: mifepristone; CCK-8: cholecystokinin-8. FIG. 2B shows a reduction in serum corticosterone concentrations after social interaction in GF mice injected with MET. N=5 SPF-CMC, 5 GF-CMC, 6 SPF-MET, 5 GF-MET mice per group. FIG. 2C depicts social activity tested using the RSI paradigm in SPF and GF test mice injected with CMC or MET (subject), in the context of SPF novel mice. N=5 SPF-CMC, 5 GF-CMC, 6 SPF-MET, 5 GF-MET mice per group (subject only). FIG. 2D shows reduction in serum corticosterone concentrations in adrenalectomized mice with ABX treatment, compared to sham control. N=11 vehicle sham, 18 ABX sham, 11 ABX ADX mice per group. FIG. 2E shows social activity tested using the RSI paradigm in vehicle sham, ABX sham, and ABX ADX test mice injected with CMC, RU-486, or MET (subject), in the context of SPF novel mice. N=11 vehicle sham, 18 ABX sham, 11 ABX ADX mice per group (subject only). FIG. 2F shows social activity tested using the RSI paradigm in vehicle sham, ABX sham, and ABX SDV test mice (subject), in the context of SPF novel mice. N=7 vehicle sham, 9 ABX sham, 6 ABX SDV mice per group (subject only). Data represent mean±SEM. Data analyzed by two-way ANOVA with Bonferroni's multiple comparison post-hoc test (FIG. 2B, FIG. 2C, FIG. 2E) and one-way ANOVA with Bonferroni's multiple comparison post-hoc test (FIG. 2D, FIG. 2F). *p≤0.05; **p≤0.01; ***p≤0.001; ****p≤0.0001.

FIGS. 3A-3N depict rescue of microbiome-mediated social deficits by knockout of glucocorticoid receptors in specific brain regions. FIG. 3A depicts a timeline scheme of stereotaxic surgery, ABX treatment, drug administration, social behavior, and sample collection. CMC: carboxymethylcellulose (vehicle); ICI: intracerebral injection; Iso: single housing; RSI: reciprocal social interaction; RU-486: mifepristone. FIG. 3B shows a diagram of viruses injected into the DG of Nr3cl^{f/f} mice (Nr3cl^{ΔDG}) to ablate glucocorticoid receptors locally. FIG. 3C shows social activity tested using the RSI paradigm in ABX test mice (subject), in the context of SPF novel mice. N=7 mice per group (subject only). FIG. 3D shows a reduction in serum corticosterone concentrations after social interaction in ABX Nr3cl^{ΔDG} mice. N=7 mice per group. FIG. 3E depicts quantification of c-Fos+ cells in various brain regions of ABX Nr3cl^{ΔDG} and control mice. N=7 mice per group. FIG. 3F shows diagram of viruses injected into the BNST of Nr3cl^{f/f} mice (Nr3cl^{ΔBNST}) to ablate glucocorticoid receptors locally. FIG. 3G shows social activity tested using the RSI paradigm in ABX test mice (subject) in the context of SPF novel mice. N=7 mice per group (subject only). FIG. 3H shows reduction in serum corticosterone concentrations after social interaction in ABX Nr3cl^{ΔBNST} mice. N=7 mice per group. FIG. 3I shows quantification of c-Fos+ cells in various brain regions of ABX Nr3cl^{ΔBNST} and control mice. N=7 mice per group. FIG. 3J shows diagram of viruses injected into the hypothalamus of Nr3cl^{f/f} mice)(Nr3cl^{ΔHYPO}) to ablate glucocorticoid receptors locally. FIG. 3K shows social activity tested using the RSI paradigm in ABX test mice (subject), in the context of SPF novel mice. N=6 control, 5 Nr3cl^{ΔHYPO} mice per group (subject only). FIG. 3L shows elevation of serum corticosterone concentrations after social interaction in ABX Nr3ck^{ΔHYPO} mice. N=6 control, 5 Nr3cl^{ΔHYPO} mice per group. FIG. 3M depicts quantification of c-Fos+ cells in various brain regions of ABX Nr3cl^{ΔHYPO} and control mice. N=6 control, 5 Nr3cl^{ΔHYPO} mice per group. FIG. 3N shows measurement of neuronal activity by c-Fos staining of various brain sections. Representative images of c-Fos staining after social interaction in PVN, adBNST, and DG in Nr3cl^{ΔDG}, Nr3cl^{ΔBNST}, Nᵣ3cl^{ΔHYPO}, and their corresponding control groups. Scale bar=100 μm. LV: lateral ventricle; 3V: third ventricle. Data represent mean±SEM. Data analyzed by two-way ANOVA with Bonferroni's multiple comparison post-hoc test (FIG. 3C, FIG. 3G, FIG. 3K) and two-tailed unpaired t-test (FIG. 3D, FIG. 3E, FIG. 311, FIG. 31, FIG. 3L, FIG. 3M). * p <0.05; **p <0.01; ***p <0.001; ****p <0.0001.

FIGS. 4A-4K show that inactivation of CRH neurons in the PVN reverses the social deficit in mice devoid of a gut microbiome. FIG. 4A depicts an analysis of gene for the CRH gene expression of hypothalamus. N=6 vehicle mice without social exposure, 6 ABX mice without social exposure, 5 vehicle mice with social exposure 6 ABX mice with social exposure per group. FIG. 4B depicts a timeline schematic of stereotaxic surgery, ABX treatment, drug administration, social behavior, and sample collection. CNO: clozapine N-oxide; ICI: intracerebral injection; Iso: single housing; ROB: retro-orbital blood collection; RSI: reciprocal social interaction. FIG. 4C shows a diagram of hM4Di-mCherry or mCherry virus injection into the PVN of Crh-ires-Cre. FIG. 4D shows social activity tested using the RSI paradigm in ABX test mice (subject), in the context of SPF novel mice. N=10 mCherry, 11 hM4Di mice per group (subject only). FIG. 4E shows non-social activity recorded using the RSI paradigm in ABX mice (subject), in the context of SPF novel mice. N=10 mCherry, 11 hM4Di mice per group (subject only). FIG. 4F shows a reduction in serum corticosterone concentrations after social interaction in ABX hM4Di mice when injected with CNO. N=9 mCherry, 10 hM4Di mice per group. FIG. 4G shows quantification of c-Fos+ cells in various brain regions of ABX hM4Di and mCherry mice. N=10 mCherry, 11 hM4Di mice per group. (FIGS. 41I-4K) depict measurement of neuronal activity by c-Fos staining of brain sections. Representative images of c-Fos, mCherry and DAPI staining in the PVN (FIG. 411, FIG. 41), adBNST (FIG. 4J) and DG (FIG. 4K) in hM4Di and mCherry mice upon CNO injection after social interaction. Scale bar=100 μm. LV: lateral ventricle; 3V: third ventricle. (FIGS. 4D-4K) All mice received antibiotics. Data represent mean±SEM. Data analyzed by two-way ANOVA with Bonferroni's multiple comparison post-hoc test (FIG. 4A, FIG. 4D, FIG. 4E) two-tailed paired t-test (FIG. 4F), and two-tailed unpaired t-test (FIG. 4G). *p≤0.05; **p≤0.01; ***p≤0.001; ****p≤0.0001, ND: no difference.

FIGS. 5A-5K depict inducement of social impairment by activation of CRH neurons and agonism of glucocorticoid receptors. FIG. 5A shows timeline schematic of stereotaxic surgery, drug administration, social behavior, and sample collection. Crh-ires-Cre mice were stereotaxically injected with viruses into the PVN at 7-8 weeks of age. Social behavior was tested under the vehicle (VEH) or clozapine N-oxide (CNO) injection. ICI: intracerebral injection; Iso: single housing; ROB: retro-orbital blood collection; RSI: reciprocal social interaction. Diagram of viruses injected into the PVN of Crh-ires-Cre mice to deliverhM3Dq-mCherry or mCherry. FIG. 5B shows serum corticosterone concentrations in SPF mCherry (left) and SPF hM3Dq (right) mice after social interaction when injected with CNO. N=10 mice per group. FIG. 5C depicts social activity tested using the RSI paradigm in SPF test mice (subject), in the context of SPF novel mice. N=10 mCherry, 11 hM3Dq mice per group (subject only). FIG. 5D depicts a timeline schematic of guide cannula stereotaxic surgery, intracerebral injection of artificial cerebrospinal fluid (ACSF) and corticotropin releasing factor peptide (CRF), social behavior, and sample collection. Wild-type (C57BL/6J) SPF mice were stereotaxically implanted with guide cannulas in the PVN at 8 weeks of age. One week post-operation, social behavior was tested following ACSF or CRF administration. Diagram of guide cannula implanted into the PVN of wild-type SPF mice to deliver CRF or ACSF. FIG. 5E and FIG. 5F shows social activity tested using the RSI paradigm in SPF test mice (subject), in the context of SPF novel mice. N=9 high CRF, 7 low CRF mice per group (subject only). FIG. 5G depicts a timeline schematic of guide cannula stereotaxic surgery, intracerebral injection of vehicle, corticosterone (CORT) and dexamethasone (DEX), social behavior, and sample collection. FIG. 511 and FIG. 5J shows a diagram of guide cannula implanted into the DG (FIG. 511) and BNST (FIG. 5J) of wild-type SPF mice to deliver vehicle, CORT, or DEX. FIG. 5I and FIG. 5K shows social activity using the RSI paradigm in SPF test mice (subject), in the context of SPF novel mice. N=7 mice per group (subject only). (FIG. 5K) N=8 vehicle, 8 CORT, 6 DEX mice per group (subject only). Data represent mean±SEM. Data analyzed by two-way ANOVA with Bonferroni's multiple comparison post-hoc test (FIG. 5C), two-tailed paired t-test (FIG. 5B, FIG. 5E, FIG. 5F, FIG. 5I, FIG. 5K). **p≤0.01; ***p≤0.001; ****p≤0.0001; ND: no difference.

FIGS. 6A-6L show restoration of social deficits and corticosterone levels by Enterococcus faecalis in mice. FIG. 6A Left: depicts a timeline schematic of ABX treatment and social behavior testing in mice. Iso: single housing; RSI: reciprocal social interaction; A: ampicillin; V: vancomycin; N: neomycin; M: metronidazole. Relevant to (FIG. 6B, FIG. 6C, FIG. 6F). Right: Timeline schematic for colonization of GF recipient mice with microbiota from ABX-treated donors (generating “Ex-GF” mice) and social behavior testing. GF: germ-free (GF). Relevant to (FIG. 6D, FIG. 6E, FIG. 6G). FIG. 6B depicts social activity tested using the RSI paradigm in AVNM, VNM, ANM, AVM, and AVN test mice (subject), in the context of SPF novel mice. FIG. 6C shows reduction in serum corticosterone concentrations after social interaction in AVM mice compared to AVNM mice. N=8 AVNM, 7 AVM mice per group. FIG. 6D shows social activity tested using the RSI paradigm in the context of SPF novel mice where GF recipient mice received microbiota via fecal transplant from AVNM or AVM donor mice (subject). N=12 GF-AVNM, 14 GF-AVM mice per group (subject only). FIG. 6E shows reduction in serum corticosterone concentrations after social interaction in GF recipient mice that received AVM microbiota, compared to GF recipient mice that received AVNM microbiota. N=12 GF-AVNM, 14 GF-AVM mice per group. FIG. 6F shows percent composition of bacteria in feces from AVNM and AVM treated donor mice at the family level, depicted as bar plots. The top 8 most abundant taxa are shown. FIG. 6G depicts Enterococcus loads analyzed in fecal pellets from mice receiving FMT either from AVM or AVNM treated donor mice. All measurements performed with digital PCR and normalized to fecal pellet weights. The red dashed line represents the lower limit of quantification (LLOQ) in the dPCR assay. FIG. 6H shows a timeline schematic depicting two rounds of RSI (1st=baseline 3-week ABX effects, and 2nd=3-week Enterococcus faecalis (E.f) colonization effects). ABX mice were colonized with E. faecalis (ABX+E.f.). Control buffer (Ctrl; 1.5% sodium bicarbonate in PBS) was given to vehicle (VEH+Ctrl) mice and ABX mice (ABX+Ctrl). RSI: reciprocal social interaction; 1st: first RSI; 2nd: second RSI. FIG. 6I shows social activity tested using the RSI paradigm in VEH and ABX mice (subject), in the context of SPF novel mice in the 1st RSI test. N=8 VEH, 15 ABX mice per group (subject only). FIG. 6J shows social activity tested using the RSI paradigm in VEH+Ctrl (left), ABX+Ctrl (middle), and ABX mice colonized with E.f for 3 weeks (ABX+E.f.) (right), in the context of SPF novel mice. N=8 VEH, 8 ABX, 7 ABX+E.f. mice per group (subject only). FIG. 6K shows social activity tested following 3 weeks of E. faecalis colonization or control buffer gavage using the RSI paradigm in VEH+Ctrl, ABX+Ctrl, and ABX+E.f. mice (subject) in the context of SPF novel mice. N=8 VEH+Ctrl, 8 ABX+Ctrl, 7 ABX+E.f. mice per group (subject only). Note, ABX+Ctrl mice are half the same group used in (FIG. 6I), and thus show a baseline reduction in social activity compared to vehicle controls. FIG. 6L shows elevation in serum corticosterone concentrations in ABX+Ctrl mice after 2nd RSI, while colonization of E.f. decreases corticosterone levels. N=7 VEH+Ctrl, 8 ABX+Ctrl, 7 ABX+E.f. mice per group. Data represent mean±SEM. Data analyzed by one-way ANOVA with Bonferroni's multiple comparison post-hoc test (FIG. 6B, FIG. 6K, FIG. 6L), two-tailed unpaired t-test (FIGS. 6C-6E, FIG. 6I), and two-tailed paired t-test (FIG. 6J). *p≤0.05; **p≤0.01; ***p≤0.001; ****p≤0.0001; ND: no difference.

### DETAILED DESCRIPTION

In the following detailed description, reference is made to the accompanying drawings, which form a part hereof In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

Without being limited by any theory, it is contemplated that the human microbiome is altered in several neuropsychiatric disorders associated with changes in sociability (Mayer, 2014). In some embodiments, method of treating a subject with a composition comprising an effective amount of one or more Enterococcus bacteria is provided, to treat one or more symptoms of social behavioral deficit. In some embodiments, a sole active ingredient administered to the subject in the method consists essentially of the one or more Enterococcus bacteria. In some embodiments, the effective amount of the one or more Enterococcus bacteria is in a composition substantially free of bacteria other than the Enterococcus bacteria. In some embodiments, the one or more Enterococcus bacteria comprises E. faecalis. In some embodiments, a sole active ingredient administered to the subject in the method consists essentially of E. faecalis. In some embodiments, the effective amount of the one or more Enterococcus bacteria is in a composition substantially free of bacteria other than the E. faecalis.

In some embodiments, the corticosterone level in a subject in need of treatment is determined and adjusted to improve behavioral performance in the subject. The subject in need of treatment can be a subject suffering from anxiety, ASD, or a pathological condition with one or more of the symptoms of behavioral deficit. In some embodiments, the corticosterone level in a subject is detected and compared with a reference corticosterone level in a normal subject.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, N.Y. 1994); Sambrook et al., Molecular Cloning, A Laboratory Manual, Cold Springs Harbor Press (Cold Springs Harbor, N.Y. 1989). For purposes of the present disclosure, the following terms are defined below.

As used herein, the term “subject in need of the treatment” refers to a subject expressing or suffering from one or more of the behavioral disorder/symptoms mentioned above. An appropriately qualified person is able to identify such an individual in need of treatment using standard behavioral testing protocols/guidelines. The same behavioral testing protocols/guidelines can also be used to determine whether there is improvement to the individual's disorder and/or symptoms.

As used herein, the term “improvement in behavioral performance” refers prevention or reduction in the severity or frequency, to whatever extent, of one or more of the behavioral disorders, symptoms and/or abnormalities expressed by individual suffering from anxiety, ASD, or a pathological condition with one or more of the symptoms of behavioral deficit. Non-limiting examples of the behavioral symptom include repetitive behavior, decreased prepulse inhibition (PPI), and increased anxiety. The improvement is either observed by the individual taking the treatment themselves or by another person (medical or otherwise).

As used herein, the term “subject in need of the treatment” refers to a subject expressing or suffering from one or more of the behavioral disorder/symptoms mentioned above. In some embodiments, the subject in need of treatment suffers from at least one of schizophrenia, ASD, anxiety or depression. An appropriately qualified person is able to identify such an individual in need of treatment using standard behavioral testing protocols/guidelines. The same behavioral testing protocols/guidelines can also be used to determine whether there is improvement to the individual's disorder and/or symptoms.

As used herein, the term “treatment” refers to a clinical intervention made in response to a disease, disorder or physiological condition manifested by a patient, particularly a patient exhibiting behavioral deficit symptoms. The aim of treatment may include, but is not limited to, one or more of the alleviation or prevention of symptoms, slowing or stopping the progression or worsening of a disease, disorder, or condition and the remission of the disease, disorder or condition. In some embodiments, “treatment” refers to both therapeutic treatment and prophylactic or preventative measures. Those in need of treatment include those already affected by a disease or disorder or undesired physiological condition as well as those in which the disease or disorder or undesired physiological condition is to be prevented. For example, in some embodiments treatment may improve behavioral performance of the subject, including ASD-related behaviors.

“Pharmaceutically acceptable” carriers are ones which are nontoxic to the cell or mammal being exposed thereto at the dosages and concentrations employed. “Pharmaceutically acceptable” carriers in accordance with methods and uses and compositions and kits herein can comprise, but not limited to, organic or inorganic, solid or liquid excipients which is suitable for the selected mode of application such as oral application or injection, and administered in the form of a conventional pharmaceutical preparation, such as solid such as tablets, granules, powders, capsules, and liquid such as solution, emulsion, suspension and the like. Often the physiologically acceptable carrier is an aqueous pH buffered solution such as phosphate buffer or citrate buffer. The physiologically acceptable carrier may also comprise one or more of the following: antioxidants including ascorbic acid, low molecular weight (less than about 10 residues) polypeptides, proteins, such as serum albumin, gelatin, immunoglobulins; hydrophilic polymers such as polyvinylpyrrolidone, amino acids, carbohydrates including glucose, mannose, or dextrins, chelating agents such as EDTA, sugar alcohols such as mannitol or sorbitol, salt-forming counterions such as sodium, and nonionic surfactants such as TWEEN™ surfactant, polyethylene glycol (PEG), and PLURONICS™ surfactant. Auxiliary, stabilizer, emulsifier, lubricant, binder, pH adjustor controller, isotonic agent and other conventional additives may also be added to the carriers.

The pharmaceutically acceptable or appropriate carrier in accordance with methods and uses and compositions and kits herein may include other compounds known to be beneficial to an impaired situation of the GI tract (e.g., antioxidants, such as Vitamin C, Vitamin E, Selenium or Zinc); or a food composition. The food composition can be, but is not limited to, milk, yoghurt, curd, cheese, fermented milks, milk based fermented products, ice-creams, fermented cereal-based products, milk-based powders, infant formulae, tablets, liquid bacterial suspensions, dried oral supplement, or wet oral supplement.

As used herein, the term “probiotic” refers to live microorganisms, which, when administered in adequate amounts, confer a health benefit on the host. The probiotics in accordance with methods and uses and compositions and kits herein may be available in foods and dietary supplements (for example, but not limited to capsules, tablets, powders, and liquids). Non-limiting examples of foods containing probiotic include dairy products such as yogurt, fermented and unfermented milk, smoothies, butter, cream, hummus, kombucha, salad dressing, miso, tempeh, nutrition bars, and some juices and soy beverages. In some embodiments, the probiotic comprises a single microorganism. In some embodiments, the probiotic comprises a combination of microorganisms. In some embodiments, the probiotic comprises a single composition. In some embodiments, the probiotic comprises two or more compositions, which can be used together, for example administered simultaneously or administered sequentially. It is noted that a probiotic can serve as the “active ingredient” or a composition or compositions for use in administration to a subject. That is, the method, use, and/or composition or compositions (either individually or in the aggregate) can comprise an effective amount of probiotic to improve at least one behavior in a subject. In some embodiments, the probiotic is the sole active ingredient for administration to the subject. In some embodiments, the “sole active ingredient” probiotic for administration to the subject can be provided in a composition or in a method or use that is substantially free of or free of bacteria other than the probiotic, antibiotics, and drugs. Even if the probiotic is the “sole” active ingredient, the composition or composition comprising the probiotic may comprise additional substances (such as buffers, bacterial feedstock, excipients, flavors, and/or food) that do not substantially affect the behavior of the subject, but may be useful for the function of the probiotic or its administration.

In some embodiments, the probiotic is comprised in a composition or compositions that are substantially free of bacteria (other than the probiotic) and/or drugs or antibiotics. By “substantially free” or “substantially absent”, it is understood that while bacteria other than the probiotic, drug, and/or antibiotic may be present in trace amounts, the bacteria other than the probiotic, drug, and/or antibiotic have no appreciable effect on the subject.

As used herein “effective amount” of probiotic refers to a quantity sufficient to achieve a clinically significant change in a behavior of a subject.

As used herein, the term “nutraceutical” refers to a food stuff (as a fortified food or a dietary supplement) that provides health benefits. Nutraceutical foods are not subject to the same testing and regulations as pharmaceutical drugs.

### Probiotics for Treatment of Social Deficit

Without being limited by any theory, it is contemplated herein that the gut microbiome can impact social interactions via discrete neuronal circuits that mediate stress responses in the brain. Bidirectional communication between the gut and the brain impacts health and disease (Dinan, 2015; Mayer, 2014; Schroeder, 2016; Rogers, 2016; Needham, 2018). Gastrointestinal (GI) physiology is affected by neural signals arising locally within the gut and emanating from the central nervous system (CNS). Reciprocally, neuroactive molecules, immune signaling, and hormones produced in the gut can impact brain function (Mayer, 2014). While several environmental and/or peripheral factors influence gut-brain interactions, the intestinal microbiota (100 trillion symbiotic bacteria, fungi, and archaea) contributes to brain development and function (Sharon, 2016). Germ-free and antibiotic-treated mice display marked differences in brain morphology, transcriptional patterns, neurogenesis, and expression of neurotransmitters, neuropeptides, and synaptic activity-related molecules (Diaz Heijtz, 2011; Sudo, 2004; Bercik, 2011; Arentsen, 2015; Gareau, 2011; Hoban, 2017; Hoban, 2016; Stilling, 2015; Zeng, 2016; Luczynski, 2016; Mohle, 2016; Ogbonnaya, 2015). Moreover, the absence of gut bacteria impacts behavior by altering stress responses, anxiety, locomotion, and social behavior (Diaz Heijtz, 2011; Sudo, 2004; Clarke, 2013; Neufeld, 2011; Desbonnet, 2014). Specific gut bacterial species modulate various complex behaviors in mice including anxiety, vocalization, repetitive behaviors and sensorimotor gating (Bravo, 2011; Hsiao, 2013).

Social interaction among animals is critical for essential traits such as survival, feeding, defense, and mating (Anderson, 2016). Impaired social communication is a hallmark of many neuropsychiatric disorders, including autism spectrum disorder (ASD), schizophrenia, anxiety, and depression(American Psychiatric Association, 2013). Social behavior is associated with the internal brain state of the subject, and complex social interactions are regulated by a series of intricate processes within dedicated regions of the brain (Anderson, 2016). Three components are crucial for the control of social behavior: sensory processing, internal states, and decision-making (Chen, 2018). Initially, an animal perceives visual, olfactory, pheromonal, auditory, and/or tactile cues from another subject. These diverse cues may represent factors modulating the internal state of the animal toward a decision on a particular social behavior. In addition, past experiences, emotions, motivation, and physiological inputs shape the internal state, influencing the final outcome of a social response (Chen, 2018). Most research to date has investigated mechanisms underlying olfactory and pheromonal-dependent social behaviors. Key brain regions, such as the amygdala, bed nucleus of stria terminalis (BNST), hypothalamus, and midbrain are involved in social circuits (Anderson, 2016; Chen, 2018). Activation or inactivation of specific brain regions or circuits can determine the quality of responses toward another subject, such as a decision to mate, nurture, fight, surrender, flee, or investigate (Anderson, 2016; Chen, 2018).

In addition to central effects originating in the CNS, evidence suggests that the regulation of social behavior may be influenced by the GI tract (Needham, 2018). Diet choice appears to impact social aggression (Hanstock, 2004). Most, but not all (Arentsen, 2015), studies show that rodents devoid of gut bacteria display decreased social behavior compared to animals with a complex microbiome (Desbonnet, 2014; Buffington, 2016; Crumeyrolle-Arias, 2014), largely characterized by reduced interest in social interaction and impaired social memory. Mouse models of ASD display changes in gut microbiome composition, which may be associated with impaired social communication, with evidence of probiotic treatment correcting certain social outcomes (Hsiao, 2013; Buffington, 2016; De Palma, 2015; Golubeva, 2017; Gacias, 2016). In humans, GI co-morbidities such as diarrhea, constipation, abdominal pain and increased intestinal permeability have been observed in several psychiatric disorders with a social component (Gorrindo, 2012; Buie, 2010), as well as altered microbiomes compared to healthy controls (Kang, 2013; Adams, 2011).

Functional magnetic resonance imaging (fMRI) reveals healthy humans differ in activity within brain regions associated with social behaviors based on their microbiome profile (Tillisch, 2017). Despite the emerging appreciate for gut bacterial effects on complex behavior, the identity of neural circuits co-opted by the microbiome to influence social activity remain unknown.

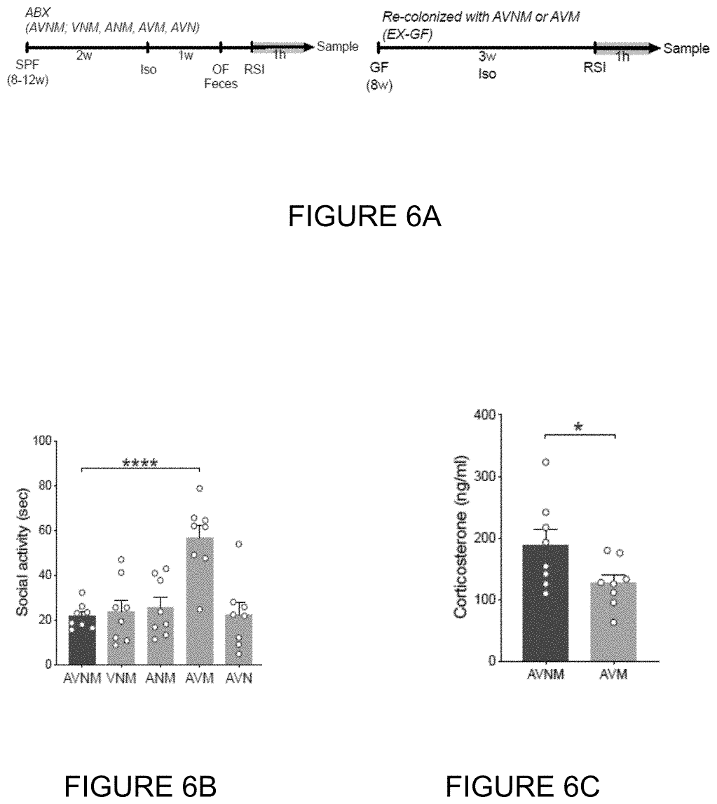

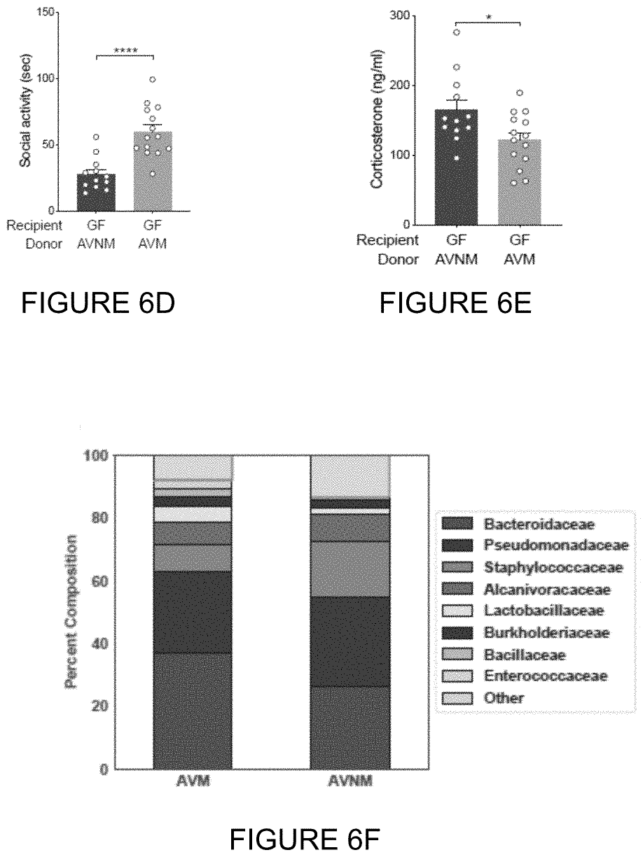

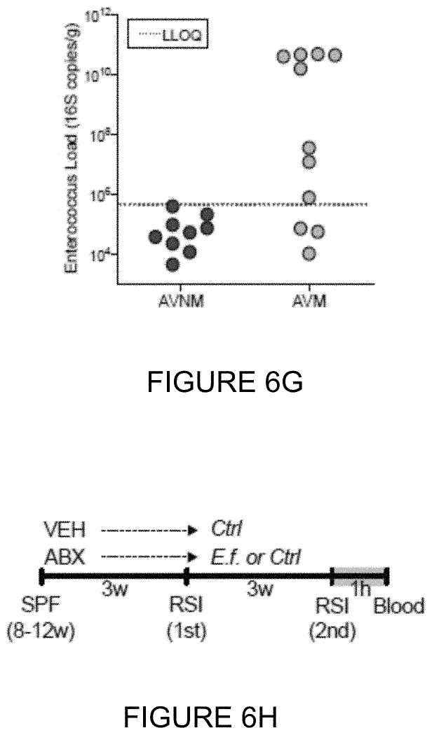

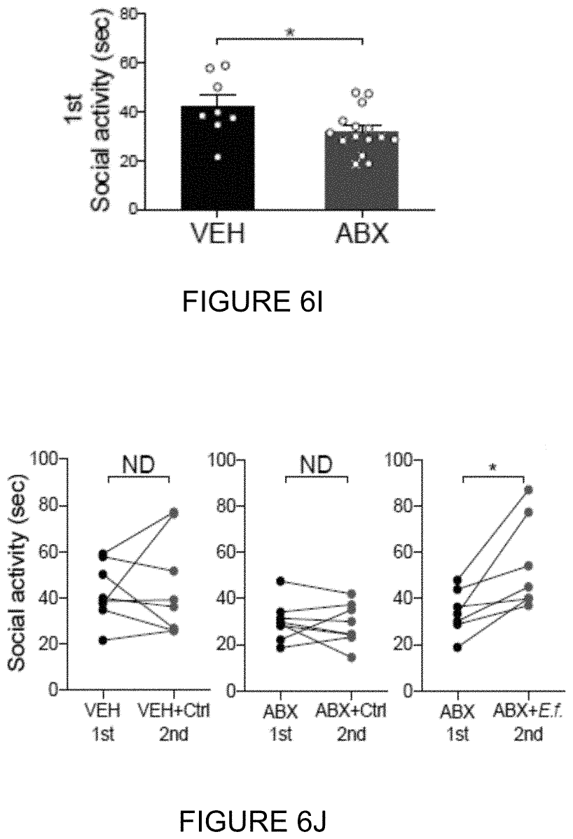

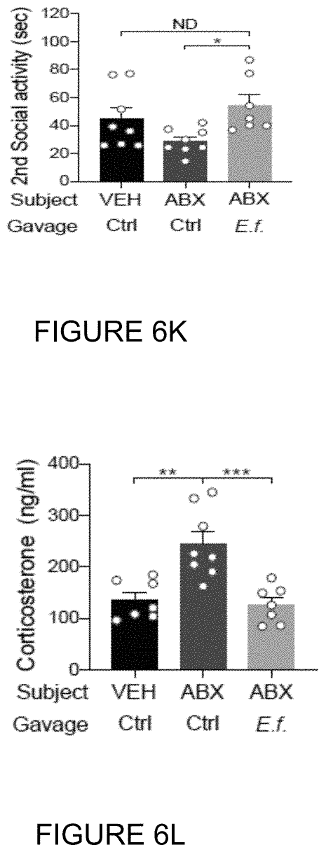

Enterococcus faecalis has been observed to restore social deficits. It has been shown that treatment with E. faecalis restores social deficits in mice, whereas treatment with vehicle has no significant effect (see FIGS. 6H-6L). Accordingly, in some embodiments described herein, probiotic treatments for social deficits are provided. The treatment can comprise administering a probiotic comprising, consisting essentially of, or consisting of E. faecalis to the subject. The subject can be in need of improvement in social behavior.

Following administration of the bacteria, social behavior can be improved. Optionally, the subject is administered no other bacteria, or substantially no other bacteria apart from the identified bacteria of the probiotic, and as such the probiotic for use in treatment of the subject is in a composition or compositions free or substantially free of other bacteria. Optionally, the subject is administered no antibiotics, or is administered substantially no antibiotics, and as such the probiotic for administration to the subject is in a composition or compositions free or substantially free of antibiotics. Optionally, the subject is administered no drugs, or is administered substantially no drugs, and as such the probiotic for administration to the subject is in a composition or compositions free or substantially free of drugs. Optionally, the subject is administered no pharmaceutically active ingredients, or is administered substantially no pharmaceutically active ingredients, and as such the probiotic for administration to the subject is in a composition or compositions free or substantially free of pharmaceutically active ingredients. In some embodiments, the subject is in need of improved social behavior, and following administration of the probiotic, social behavior is improved. In some embodiments, the behavior in need of improvement (and subsequently improved) comprises at least one of communication, sociability, or language comprehension and/or language production. In some embodiments, the subject has ASD. In some embodiments, the method further comprises determining whether the subject has ASD, and the effective amount of probiotic is administered if the subject has ASD. In some embodiments, the subject has schizophrenia. In some embodiments, the method further comprises determining whether the subject has schizophrenia, and the effective amount of probiotic is administered if the subject has schizophrenia. In some embodiments, the subject has anxiety. In some embodiments, the method further comprises determining whether the subject has anxiety, and the effective amount of probiotic is administered if the subject has anxiety. In some embodiments, the subject has depression. In some embodiments, the method further comprises determining whether the subject has depression, and the effective amount of probiotic is administered if the subject has depression.

In accordance with any of the embodiments described above, optionally, each composition, use or method is free of, or is substantially free of bacteria other than the identified bacteria of the probiotic. In accordance with any of the embodiments above, optionally, each composition is free of, or is substantially free of antibiotics. In accordance with any of the embodiments above, optionally, each composition is free of, or is substantially free of bacteria other than the probiotic and antibiotics.

In accordance with embodiments described herein, the probiotics of the methods, uses, and compositions described herein can be for any suitable route of administration. For example, the probiotic can be administered to the subject via oral administration, rectum administration, transdermal administration, intranasal administration or inhalation. In some embodiments, the probiotic is administered to the subject orally.

In some embodiments, the effective amount of bacteria in the probiotic composition, use, or method includes at least about 10⁴ colony forming units (cfu), for example at least about 10⁴, 10⁵, 10⁶, 10⁷, 10⁸, 10⁹, 10¹⁰, 10¹¹, 10¹², or 10¹³ cfu, including ranges between any of the listed values, for example 10⁴-10⁸ cfu, 10⁴-10⁹ cfu, 10⁴-10¹⁰ cfu, 10⁴-10¹¹ cfu, 10⁴-10¹² cfu, 10⁴-10¹³ cfu, 10⁵-10⁸ cfu, 10⁵-10⁹ cfu, 10⁵-10¹⁰ cfu, 10⁵-10¹¹ cfu, 10⁵-10¹² cfu, 10⁵-10¹³ cfu, 10⁶-10⁸ cfu, 10⁶-10⁹ cfu, 10⁶-10¹⁰ cfu, 10⁶-10¹¹ cfu, 10⁶-10¹² cfu, 10⁶-10¹³ cfu, 10⁷-10⁸ cfu, 10⁷-10⁹ cfu, 10⁷-10¹⁰ cfu, 10⁷-10¹¹ cfu, 10⁷-10¹² cfu, 10⁷-10¹³ cfu, 10⁸-10⁹ cfu, 10⁸-10¹⁰ cfu, 10⁸-10¹¹ cfu, 10⁸-10¹² cfu, or 10⁸-10¹³ cfu. In some embodiments, the effective amount of bacteria comprises a log phase quantity (at 37° C.) of bacteria in a composition for administration to the subject. In some embodiments, the effective amount of bacteria comprises a stationary phase quantity (at 37° C.) of bacteria in a composition for administration to the subject.

### Methods of Treating Social Deficit Symptoms

In some embodiments, methods of treating social deficit symptoms are provided. The method can comprise identifying a subject as in need of improving a social behavior. The method can comprise administering an effective amount of a probiotic comprising, consisting essentially of, or consisting of Enterococcus bacteria as described herein to the subject in need of improved social behavior. In some embodiments, the subject is in need of improved social behavior, and following administration of the probiotic, social behavior is improved. In some embodiments, the behavior in need of improvement (and subsequently improved) comprises at least one of communication, repetitive behavior, decreased prepulse inhibition (PPI), and increased anxiety, sociability, or language comprehension and/or language production. In some embodiments, the subject has ASD. In some embodiments, the method further comprises determining whether the subject has ASD, and the effective amount of probiotic is administered if the subject has ASD. In some embodiments, the subject has schizophrenia. In some embodiments, the method further comprises determining whether the subject has schizophrenia, and the effective amount of probiotic is administered if the subject has schizophrenia. In some embodiments, the subject has anxiety. In some embodiments, the method further comprises determining whether the subject has anxiety, and the effective amount of probiotic is administered if the subject has anxiety. In some embodiments, the subject has depression. In some embodiments, the method further comprises determining whether the subject has depression, and the effective amount of probiotic is administered if the subject has depression.

In some embodiments, methods of ameliorating social deficit symptoms are provided. The method can comprise identifying the subject as in need of improving a social behavior. The method can comprise administering a probiotic comprising, consisting essentially of, or consisting of an effective amount of Enterococcus bacteria to the subject. Following acute administration of the probiotic, the social deficit symptoms can be ameliorated. In some embodiments, the subject is identified as having ASD or schizophrenia symptoms that comprise gastrointestinal abnormalities associated with ASD or schizophrenia, and these symptoms are improved following administration of the probiotic. In some embodiments, the subject is identified as having ASD or schizophrenia symptoms that comprise immunological abnormalities associated with ASD or schizophrenia, and these symptoms are improved following administration of the probiotic. In some embodiments, the subject is identified as having ASD or schizophrenia symptoms that comprise immunological and gastrointestinal abnormalities associated with ASD or schizophrenia, and these symptoms are improved following administration of the probiotic. In some embodiments, the subject is identified as having ASD symptoms that comprise gastrointestinal abnormalities associated with ASD, and these symptoms are improved following administration of the probiotic. In some embodiments, the subject is identified as having ASD symptoms that comprise immunological abnormalities associated with ASD, and these symptoms are improved following administration of the probiotic. In some embodiments, the subject is identified as having ASD symptoms that comprise immunological and gastrointestinal abnormalities associated with ASD, and these symptoms are improved following administration of the probiotic. In some embodiments, the subject is identified as having schizophrenia symptoms that comprise gastrointestinal abnormalities associated with schizophrenia, and these symptoms are improved following administration of the probiotic. In some embodiments, the subject is identified as having schizophrenia symptoms that comprise immunological abnormalities associated with schizophrenia, and these symptoms are improved following administration of the probiotic. In some embodiments, the subject is identified as having schizophrenia symptoms that comprise immunological and gastrointestinal abnormalities associated with schizophrenia, and these symptoms are improved following administration of the probiotic. In some embodiments, the subject is further identified as in need or improving sensorimotor gating, communication, anxiety, and or repetitive behavior, and following administration of the probiotic, the corresponding sensorimotor gating, communication, anxiety, and or repetitive behavior(s) are improved.

In some embodiments, methods of treating ASD or schizophrenia symptoms are provided. The method can comprise identifying the subject as being in need of improving repetitive behavior. The method can comprise administering a probiotic comprising, consisting essentially of, or consisting of an effective amount of Enterococcus bacteria as described herein to the subject. Following acute administration of the probiotic, repetitive behavior in the subject can be improved.

In some embodiments, methods of preventing ASD symptoms are provided. The method can comprise identifying a subject as at risk for developing a communication behavior deficiency or sensory gating behavior deficiency or both. The method can comprise administering an effective amount of a probiotic comprising, consisting essentially of, or consisting of Enterococcus bacteria as described herein to the subject at risk. The subject can develop with minimized deficiencies or no discernable deficiencies in social communication behavior. In some embodiments, the communication behavior comprises at least one of communication, sociability, or language comprehension and/or language production. Optionally, the at-risk subject is an infant or child.

In some embodiments, depletion of the microbiota activates or de-represses activity in distinct brain regions associated with stress responses in response to social interaction. Examples of brain regions that are activated or de-repressed include, but not limited to, hippocampal dentate gyms (DG), paraventricular nucleus of the hypothalamus (PVN), and BNST.

In some embodiments, methods of treating social deficit symptoms are provided. The method can comprise identifying a subject having high corticosterone levels or a subject who would benefit from reduced corticosterone levels. The method can comprise administering an effective amount of a probiotic comprising, consisting essentially of, or consisting of Enterococcus bacteria as described herein to the subject to regulate corticosterone levels in the subject. In some embodiments, the subject is in need of regulated corticosterone levels, and following administration of the probiotic, corticosterone levels are regulated.

Various methods can be used to regulate the corticosterone level for improving a social deficit in the subject. For example, a pharmacological corticosterone synthesis blocker can be used to effectively reduce corticosterone levels. As another example, removal of adrenal gland can be used to effectively reduce corticosterone levels. In some embodiments, pharmacological antagonization of glucocorticoid receptor can be used to effectively reduce corticosterone levels. As still yet another example, deleting the gene encoding glucocorticoid receptor can be used to effectively reduce corticosterone levels.

One of ordinary skill in the art will appreciate that variability in the level of corticosterone may exist between individuals, and a reference level can be established as a value representative of the level of corticosterone in a behaviorally normal population, or a population of subjects that do not suffer from ASD, anxiety or any pathological condition with one or more of the symptoms of social deficit, for the comparison. Various criteria can be used to determine the inclusion and/or exclusion of a particular subject in the reference population, including age of the subject (e.g. the reference subject can be within the same age group as the subject in need of treatment) and gender of the subject (e.g. the reference subject can be the same gender as the subject in need of treatment). In some embodiments, corticosterone levels are increased in the subject suffering from social deficit as compared to the reference level. In some embodiments, corticosterone levels are decreased in the subject suffering from social deficit as compared to the reference level. In some embodiments, the alteration in the corticosterone level can be restored partially or fully by adjusting the composition of gut microbiota in the subject suffering from social deficit.

Glucocorticoid receptor is involved in various signaling pathways. In some embodiments, genetic ablation of a glucocorticoid receptor in specific brain regions rescue social impairments caused by microbiome depletion. Examples of brain regions include, but not limited to, hippocampal dentate gyms (DG), paraventricular nucleus of the hypothalamus (PVN), and BNST. For example, genetic ablation of glucocorticoid receptor in DG or BNST region of the brain rescue social impairments caused by microbiome depletion. In addition, chemogenetic inactivation of neuronal populations in the paraventricular nucleus of the hypothalamus (PVN) rescue social impairments caused by microbiome depletion. For example, chemogenic inactivation of corticotrophin-releasing hormone (CRH)-expressing neurons in the PVN of mice is sufficient to induce social deficits increases social behavior in microbiome depleted mice.

In some embodiments, for any of the above methods, the probiotic comprising, consisting essentially of, or consisting of Enterococcus bacteria as described herein is administered to the subject in need of improved social behavior. Optionally, the subject is administered no other bacteria, or substantially no other bacteria apart from the identified bacteria of the probiotic, and as such the probiotic for use in treatment of the subject is in a composition or compositions free or substantially free of other bacteria. Optionally, the subject is administered no antibiotics, or is administered substantially no antibiotics, and as such the probiotic for administration to the subject is in a composition or compositions free or substantially free of antibiotics. Optionally, the subject is administered no drugs, or is administered substantially no drugs, and as such the probiotic for administration to the subject is in a composition or compositions free or substantially free of drugs. Optionally, the subject is administered no pharmaceutically active ingredients, or is administered substantially no pharmaceutically active ingredients, and as such the probiotic for administration to the subject is in a composition or compositions free or substantially free of pharmaceutically active ingredients.

In some embodiments as described above, the method further comprises determining that the subject is in need of improving a behavior. In some embodiments, for example uses, methods, and or compositions directed to infants and/or children, a subject at risk for an ASD behavior is identified based on maternal immune activation and/or other risk factors In some embodiments, the subject is determined to have a lesion or developmental deficiency in a region of the brain associated with speech production, speech recognition, impulse control, and socialization, for example regions of the cerebral cortex, the corpus colosum, Broca' s area, and/or Wernicke's area. In some embodiments, an ASD behavior, for example a deficient communication, vocalization, sensorimotor, anxiety, and/or repetitive behavior, or a combination of two or more of these is identified using standard diagnostic criteria, for example in the Diagnostic and Statistical Manual of Mental Disorders, Fourth Edition (DSM-4) or Fifth Edition (DSM-5). In some embodiments, the presence or absence of ASD in the subject is determined using a behavioral test, for example at least one of the Autism Behavior Checklist (ABC), Autism diagnostic Interview-Revised (ADI-R), childhood autism Rating Scale (CARS), and/or Pre-Linguistic Autism Diagnostic Observation Schedule (PL-ADOS). The behavioral test can include, but is not limited to, detecting the presence and/or extent of 1) preoccupation with one or more stereotyped and restricted patterns of interest that is abnormal in either intensity or focus, 2) inflexible adherence to specific, nonfunctional routines or rituals, c) stereotyped and repetitive motor mannerisms (such as hand flapping, finger flapping etc.), and/or d) persistent preoccupation with parts of objects. Non-limiting examples of behavior that can be included in a behavioral test and suggest a need for improving behavioral performance in the subject under the test include: a) sensory behaviors, including poor use of visual discrimination when learning, seems not to hear, so that a hearing loss is suspected, sometimes shows no “startle response” to loud noise”, sometimes painful stimuli such as bruises, cuts, and injections evoke no reaction, often will not blink when bright light is directed toward eyes, covers ears at many sounds, squints, frowns, or covers eyes when in the presence of natural light, frequently has no visual reaction to a “new” person, stares into space for long periods of time; b) relating behaviors: frequently does not attend to social/environmental stimuli, has no social smile, does not reach out when reached for, non-responsive to other people's facial expressions/feelings, actively avoids eye contact, resists being touched or held, is flaccid when held in arms, is stiff and hard to held, does not imitate other children at play, has not developed any friendships, often frightened or very anxious, “looks through” people; c) body and object use behaviors: whirls self for long periods of time, does not use toys appropriately, insists on keeping certain objects with him/her, rocks self for long periods of time, does a lot of lunging and darting, flaps hands, walks on toes, hurts self by banging head, biting hand, twirls, spins, and bangs objects a lot, feel, smell, and/or taste objects in the environment, gets involved in complicated “rituals” such as lining things up, is very destructive; and d) language behaviors: does not follow simple commands given once, has pronoun reversal, speech is atonal, does not respond to own name when called out among two others, seldom says “yes” or “I”, does not follow simple commands involving prepositions, gets desired objects by gesturing, repeats phrases over and over, cannot point to more than five named objects, uses 0-5 spontaneous words per day to communicate wants and needs, repeats sounds or words over and over, echoes questions or statements made by others, uses at least 15 but less than 30 spontaneous phrases daily to communicate, learns a simple task but “forgets” quickly, strong reactions to changes in routine/environment, has “special abilities” in one area of development, which seems to rule out mental retardation, severe temper tantrums and/or frequent minor tantrums, hurts others by biting, hitting, and/or kicking, does not wait for needs to be met, difficulties with toileting, does not dress self without frequent help, frequently unaware of surroundings, and may be oblivious to dangerous situations, prefers to manipulate and be occupied with inanimate things, and/or a developmental delay identified at or before 30 months of age. One of ordinary skill in the art would appreciate that the attending physician would know how to identify a subject in need of treatment disclosed herein.

In some embodiments as described above, the method comprises administering the effective amount of probiotic in a single administration of one or more compositions. In some embodiments as described above, the method comprises administering the effective amount of the probiotic across two or more administrations of a single composition as described herein. For example, the compositions can be administered about 1 minute, 2, 3, 4, 5, 6, 7, 8, 9, 10, 15, 20, 25, 30, 35, 40, 45, 50, 55 minutes, 1 hour, 2, 3, 4, 5, 6, 7, 8, 9, 10, 11, 12, 13, 14, 15, 16, 17, 18, 19, 20, 21, 22, 23 hours, 1 day, 2, 3, 4, 5, 6, 7, 8, 9, or 10 days apart, including ranges between any two of the listed values, for example 1 minute-10 minutes, 1 minute to 30 minutes, 1 minute to 1 hour, 1 minute-2 hours, 1 minute-4 hours, 1 minute-12 hours, 1 minute-18 hours, 1 minute-1 day, 10 minutes to 30 minutes, 10 minutes to 1 hour, 10 minutes-2 hours, 10 minutes-4 hours, 10 minute-12 hours, 10 minutes-18 hours, 10 minutes-1 day, 30 minutes to 1 hour, 30 minutes-2 hours, 30 minutes-4 hours, 30 minute-12 hours, 30 minutes-18 hours, 30 minutes-1 day, 30 minutes-2 days, 1 hour-2 hours, 1 hour-4 hours, 1 hour-12 hours, 1 hour-18 hours, 1 hour-1 day, 4 hours-12 hours, 4 hours-18 hours, 4 hours-1 day, 1 day-2 days, 1 day-3 days, 1 day-4 days, 1 day-5 days, 1 day-7 days, 1 day-10 days, 2 days-3 days, 2 days-4 days, 2 days-5 days, 2 days-7 days, 2 days-10 days, or 5 days to 10 days. In some embodiments as described above, the method comprises administering the effective amount of two or more different compositions as described herein across two or more administrations of a single composition. For example, the second composition can be administered about 1 minute, 2, 3, 4, 5, 6, 7, 8, 9, 10, 15, 20, 25, 30, 35, 40, 45, 50, 55 minutes, 1 hour, 2, 3, 4, 5, 6, 7, 8, 9, 10, 11, 12, 13, 14, 15, 16, 17, 18, 19, 20, 21, 22, 23 hours, 1 day, 2, 3, 4, 5, 6, 7, 8, 9, or 10 days after the first composition, including ranges between any two of the listed values, for example 1 minute-10 minutes, 1 minute to 30 minutes, 1 minute to 1 hour, 1 minute-2 hours, 1 minute-4 hours, 1 minute-12 hours, 1 minute-18 hours, 1 minute-1 day, 10 minutes to 30 minutes, 10 minutes to 1 hour, 10 minutes-2 hours, 10 minutes-4 hours, 10 minute-12 hours, 10 minutes-18 hours, 10 minutes-1 day, 30 minutes to 1 hour, 30 minutes-2 hours, 30 minutes-4 hours, 30 minute-12 hours, 30 minutes-18 hours, 30 minutes-1 day, 30 minutes-2 days, 1 hour-2 hours, 1 hour-4 hours, 1 hour-12 hours, 1 hour-18 hours, 1 hour-1 day, 4 hours-12 hours, 4 hours-18 hours, 4 hours-1 day, 1 day-2 days, 1 day-3 days, 1 day-4 days, 1 day-5 days, 1 day-7 days, 1 day-10 days, 2 days-3 days, 2 days-4 days, 2 days-5 days, 2 days-7 days, 2 days-10 days, or 5 days to 10 days. In some embodiments, the probiotic is administered in a slow-release formulation (for example a slow-release capsule or implant) for any of the durations described above.

In some embodiments, the probiotic is administered to the subject until an improvement in behavioral performance is observed. Optionally, the probiotic is administered to the subject after an improvement in behavioral performance is observed, for example to solidify or maintain the improved behavioral performance.

As described herein, adjusting the corticosterone level can treat, inhibit, or ameliorate social deficit symptoms. As disclosed herein, amelioration is used in a broad sense to refer to at least a reduction in the magnitude of a parameter, e.g., symptom, associated with the pathological condition being treated. In some embodiments, the method can completely inhibit, e.g., prevented from happening, or stopped, e.g., terminated, such that the host no longer suffers from the pathological condition, or at least one or more of the symptoms that characterize the pathological condition. In some embodiments, the method can delay or slowing of disease progression, amelioration or palliation of the disease state, and remission (whether partial or total), whether detectable or undetectable.

### EXAMPLES

Some aspects of the embodiments discussed above are disclosed in further detail in the following examples, which are not in any way intended to limit the scope of the present disclosure.

### Experimental Material and Methods

The following experimental methods were used for Examples 1-6 described below.

### Mice

Wild-type C57BL/6J (00064), Nr3cl^{f/f} (021021; B6.Cg-Nr3cl^{tm1.1Jda}/J), Crh-ires-Cre (012704; B6(Cg)-Crh^{tm(cre)Zjh}/J), and Ai14D (007914; B6.Cg-Gt(ROSA)26Sor^{tm14(CAG-tdTomato)Hze}/J) mice were obtained through Jackson Laboratory. C57BL/6J germ-free (GF) mice were bred in the Gnotobiotic Animal Facility at Caltech. Although the data exhibited similar effects in both male and female mice, the majority of experiments were performed with male animals. All mice were group housed (2-5 mice per cage) with a 13 h light/11 h dark cycle (lights on at 06:00) at 21-23° C. and 45% relative humidity within a range of 30-70% in ventilated cages (Super Mouse 750™, Lab Products Inc). Unless specified, SPF mice were fed 5053 PicoLab Rodent Diet (LadDiet) and the SPF breeders were fed a mix of half 5053 and half 5058 PicoLab Rodent Diet. SPF C57BL/6J mice obtained from Jackson Laboratory were yielded from multiple litters (at least 6-8 litters) and randomly assigned to subject groups (SPF, vehicle, or ABX) or novel mice (SPF or ABX) to eliminate the maternal effect. GF mice used in this study were yielded from multiple litters (at least 6 litters) and randomly assigned to testing groups to eliminate the maternal effect.

For experimental mice, SPF, GF, vehicle and ABX mice were transferred and maintained in sterilized cages and fed autoclaved 5010 PicoLab Rodent Diet (LadDiet) and provided with autoclaved drinking water. GF mice were housed outside of the GF isolator for no more than a week. GF mice used in this study were tested for behavior between the age of 11 and 15 weeks. The SPF used to compare with GF were all age-matched. All GF mice completed the behavior test once and were sampled 30-60 minutes after behavior testing. The fecal samples of GF mice were freshly collected at the endpoint of each experiment. The feces were plated onto Brucella agar plates with 5% sheep's blood (B0150; Teknova) and cultured aerobically and anaerobically to screen for contamination or antibiotic resistant bacteria during sterile housing. All SPF mice were handled with the same procedures as GF mice. All experiments were performed with approval of the Caltech and NCKU Institutional Animal Care and Use Committee (IACUC).

### Antibiotic Cocktail (ABX) Treatment

Gut microbiota were depleted in adult mice (8-12 weeks) by treatment with a cocktail of antibiotics for 3-4 weeks. The recipe included drinking water with ampicillin (1 g/L), vancomycin (0.5 g/L), neomycin (1 g/L), and metronidazole (0.5 g/L), and was sweetened with 1% w/v of sucrose and filtered with a 0.22 μm filter. To avoid confounding effect from chronic stress induced by oral gavage, antibiotics were administered in the drinking water ad libitum.

Untreated (vehicle control) mice received 1% w/v of sucrose in filtered drinking water. The length of ABX treatment was adapted based on the recovery of body weight after ABX treatment. All antibiotics were United States Pharmacopeia (USP) grade, or at minimum cell culture grade. Drinking water was prepared and changed weekly. All vehicle mice went through the same handling procedures as ABX mice. Fecal samples were freshly collected at the endpoint of each experiment, and plated onto Brucella agar plates with 5% sheep's blood (B0150; Teknova) and cultured aerobically and anaerobically to examine if gut bacteria were successfully depleted following ABX treatment.

### Corticosterone Measurement

Whole blood was collected either by cardiac puncture or retro-orbital bleeding, and placed into Micro tube Z gel (41.1378.005; Sarstedt). Serum was separated by centrifuge at 10,000×g for five minutes, and stored at −80° C. until use. Corticosterone levels were detected by the Corticosterone EIA Kit (K014-H5; Arbor Assays) using the manufacturer's protocol. Due to the nature of the circadian rhythm for the corticosterone, the blood sample was collected during 1:00-5:00 pm on each day of experiment.

### Adrenalectomy

Mice were anesthetized with 5% isoflurane in an induction box followed by maintenance on a nose cone and kept at 1-2% isoflurane during surgery. A single dose of sustained-release buprenorphine (1 mg/kg) was subcutaneously injected prior to surgery. The surgical fields were covered with a sterile drape and sterile gloves were worn. A 1.5 cm dorsal midline incision was made with its midpoint centered over the 13th rib. All of the underlying muscle on either side of the spinal column was incised. The adrenal glands are located in the fat pad that covers the cranial portion of the kidney. The adrenal glands were isolated and removed using a small-curved forceps and a micro scissor. Bupivacaine (1 mg/kg) was applied subcutaneously along the incision just prior to wound closure. The peritoneal opening was closed with a 5-0 subcutaneous suture (Vicryl) and 7 mm wound clips (Roboz) were applied to close the dermis. For postoperative care, mice were monitored for signs of pain or distress at least three times a day for three days, and were supplied with drinking water containing 0.9% sodium chloride ad libitum. The clips were removed two weeks post-surgery under isoflurane anesthesia. Sham control mice underwent the same procedure as described above without removal of the adrenal glands.

### Drug Administration

To systemically inhibit corticosterone synthesis, mice were intraperitoneally injected with 50 mg/kg metyrapone (3292; R&D) and returned to their home cage. To block glucocorticoid receptors, mice were intraperitoneally injected with 40 mg/kg RU-486 (also known as mifepristone; M8046; Sigma-Aldrich) and returned to their home cage. Metyrapone and RU-486 were freshly dissolved in 0.5% carboxymethylcellulose sodium (CMC; C9481; Sigma) on the day of the reciprocal social interaction test. 0.5% CMC injection served as baseline control. Behavior testing was performed 40 minutes after injection.

For acute corticosterone exposure, mice were intraperitoneally injected on two consecutive days with 10 mg/kg corticosterone 2-hydroxypropyl-β-cyclodextrin complex (C174; Sigma-Aldrich) and returned to their home cage. The corticosterone 2-hydroxypropyl-β-cyclodextrin complex was used to facilitate solubility of the steroid. The reciprocal social interaction test was performed 40 minutes after the second corticosterone injection.

For DREADD-based chemogenetic activation or inactivation, hM3Dq, hM3Di, or mCherry expressing mice were intraperitoneally injected with 2-3 mg/kg clozapine N-oxide (CNO) (Enzo Life Sciences) and returned to their home cage. The reciprocal social interaction test was performed 32-40 minutes after the second CNO injection.

Viral vectors

AAV-hSyn-DIO-hM4Di-mCherry (44362-AAV5); AAV-hSyn-DIO-hM3Dq-mCherry (44361-AAV5); AAV-hSyn-DIO-mCherry (50459-AAV5); AAV-hSyn-EGFP (50465-AAV5) viruses were purchased through Addgene. AAV-hSyn-Cre-GFP (AAV5) was produced at the Vector core at the University of North Carolina at Chapel Hill.

### Stereotaxic Surgery

Adult 7-8 week old mice were deeply anesthetized with 5% isoflurane in oxygen and kept at 1-2% isoflurane during surgery. In addition, 5 mg/kg ketoprofen was subcutaneously given once before surgery. Surgery was performed with a stereotaxic frame (Model 1900; David Kopf Instruments). The bregma was located by using a centering scope (Model 1915; David Kopf Instruments) and aligned by a stereotaxic alignment indicator (Model 1905; David Kopf Instruments) The skull was exposed and holes were produced using a stereotaxic drill (Model 1911; David Kopf Instruments) with a #79 micro drill bit (Drill bit city). Viruses were injected into the brain in both hemispheres using a pulled glass capillary (1B120E-4; World Precision Instruments) by a nanoliter injector (Nanoliter2010; World Precision Instruments) at a flow rate of 23 nl/minute controlled by a micro controller (Micro4; World Precision Instruments). The glass capillaries were left in place for five minutes to prevent backflow. Stereotaxic injection coordinates (in millimeters): PVN (AP: −0.80, ML: ±0.25; DV: −4.75), BNST (AP: 0.26, ML: ±0.90; DV: −4.30), DG (AP: −1.50, ML: ±0.40; DV: −2.25), CeA (AP: −0.80, ML: ±2.3; DV: −4.70). Volumes delivered were 207 nl for most regions, except 299 nl for the dentate gyras (DG). Bupivacaine (1 mg/kg) was applied subcutaneously along the incision prior to wound closure.

The incision on the scalp was closed with tissue adhesive (Gluture topical adhesive; Abbott Laboratories). For postoperative care, mice were monitored daily for signs of pain or distress for at least three days and were supplied for seven days with drinking water containing 30 mg/kg ibuprofen ad libitum. Behavioral experiments were performed at least three weeks after virus injection. All surgically manipulated animals underwent histological examination after sacrifice to ensure viruses were correctly injected.

### Guide Canula Implantation Surgery

Mice were anesthetized with 5% isoflurane in oxygen in a Plexiglas cage. After anesthesia, mice were placed in a digital stereotaxic device (Stoelting) delivering isoflurane to maintain anesthesia throughout the surgery and injected subcutaneously with 5 mg/kg ketoprofen. The custom guide cannula was implanted in the following regions at the following coordinates: lateral ventricle (LV), AP: −0.1 mm; ML: 1.0 mm; DV: −2.0 mm; PVN, AP: −0.8, ML: ±0.25, DV: −4.75; DG, AP: −1.5, ML: ±0.4, DV: −2.25; BNST: AP: 0.26, ML: ±0.9, DV: −4.3. Two to four small stainless-steel screws were installed on the skull to anchor the acrylic maintaining the guide cannula. Then, a dummy stainless-steel plug was implanted in the cannula to prevent clogging of blood or cerebrospinal fluid at the cannula opening. For postoperative care, mice were monitored daily for signs of pain or distress for at least three days and were supplied for seven days with drinking water containing 30 mg/kg ibuprofen ad libitum. The customized cannulization set includes guide cannula, injector, dummy, and cap. The guide cannula was implanted 0.5 mm above the PVN, DG, and BNST. The tip of the guide cannula track can be visualized in each implanted mouse. The injector was designed to reach 0.5 mm below the tip of guide cannula. As the injector was made of 33G fine needle, the needle tracks produced by the injector were not visible in brain slices. The cannulization set for the PVN, DG, and BNST was customized (RWD Life Science) with the following specification: PVN (guide cannula: double_OD 0.41 mm-27G/C.C0.5/B7.8/M3.5/C=4.25 mm, dummy cannula: double_OD 0.20 mm-27G/C.C0.5/Mates with M3.5/G=0.5 mm, Injector: double OD0.21 mm-33G/C.C0.5/Mates with M3.5/C=4.25 mm/G=0.5 mm), DG (guide cannula: double_OD 0.41 mm-27G/C.C0.8/B7.8/M3.5/C=1.75 mm, dummy cannula: double_OD 0.20 mm-27G/C.C0.8/Mates with M3.5/G=0.5 mm, Injector: double OD0.21 mm-33G/C.C0.8/Mates with M3.5/C=1.75 mm/G=0.5 mm), BNST (guide cannula: double_OD 0.41 mm-27G/C.C1.8/B7.8/M3.5/C=3.8 mm, dummy cannula: double OD 0.20 mm-27G/C.C1.8/Mates with M3.5/G=0.5 mm, Inj ector: double OD0.21 mm-33 G/C.C1.8/Mates with M3.5/C=3.8 mm/G=0.5 mm).

### Intracerebroventricular (ICV) Antibiotics Injection

Ampicillin (1 mg/L) and metronidazole (0.5 mg/L) were dissolved in artificial cerebrospinal fluid (ACSF: 7.46 g/L NaCl, 0.19 g/L KCl, 0.14 g/L CaCl₂, 0.19g/L MgCl₂, 1.76 g/L NaHCO₃, 0.18 g/L NaH₂PO₄, 0.61 g/L glucose in ddH₂O) and adjusted to pH 7.4, respectively. The dissolved antibiotics were filtered through a 0.22 μm filter. Mice were infused with antibiotics during social behavior tests with an infusion rate of 7 nl/sec for 3 min.

### CRF Infusion in PVN

CRF was dissolved in saline (High-dose: 210 μM; Low-dose: 42 μM) and injected into each hemisphere of PVN during anesthesia with an infusion rate of 4.5 nL/sec for a total infusion volume of 450 nL. Once complete, the injection syringe remained in tissue for 2 minutes to prevent backflow. After 2 minutes, the injection syringe was placed in the other hemisphere of PVN and infused the same volume of CRF at the same flow rate. The mice were then placed in a novel cage to recover from anesthesia until social behavior testing.

### Corticosterone and Dexamethasone Infusion in DG and BNST

Corticosterone (65 μM) or dexamethasone (20 mM) was dissolved in DMSO, diluted in saline, and injected bilaterally into DG and BNST of anesthetized mice at an infusion rate of 4.5 nL/sec up to 450 nL. Once complete, the injection syringe remained in tissue for 2 minutes to prevent backflow. After 2 minutes, the injection syringe was placed in the other hemisphere of PVN and infused the same volume of corticosterone or dexamethasone at the same flow rate. The mice were then placed in a novel cage to recover from anesthesia until social behavior testing.

### Subdiaphragmatic Vagotomy (SDV)

Mice were habituated to a liquid diet (Research Diets; AIN-76A) for two days and were fasted overnight before surgery. The abdominal surgery site was shaved and wiped with the skin disinfectant Chlorhexidine three times before incision. Mice were then anesthetized using 1-5% isoflurane and injected subcutaneously with 5mg/kg ketoprofen. An incision was made along the abdominal midline and the muscle and skin were separated using blunt scissors. The liver was then gently moved using a sterile cotton swab to expose the stomach and esophagus. The ventral and dorsal trunks of the vagus nerve were resected using sharp forceps. In the sham operation, the stomach and esophagus were exposed but the vagal trunks were not resected. The organs were then placed back to their anatomical position. The incision along the muscular layer was closed by absorbable suture and treated with topical lidocaine (0.25%) and the skin incision was closed by non-absorbable suture and treated with n-butyl cyanoacrylate adhesive (3M VETBOND) to prevent infection. After surgery, the mice were placed into a clean cage placed on a heat pad. During the recovery time, mice were given a hydrating, nutritious gel pack (DietGel 76A 2 oz, Clear H₂O) for two days before receiving a normal chow diet. Mice were supplied with drinking water containing ibuprofen (20 mg/dl) ad libitum. After behavior testing, SDV was validated by intraperitoneal injection of cholecystokinin (CCK-8) following fasted-induced food consumption. Each mouse was fasted for 20 hours, placed into a single cage, and injected i.p. with CCK-8 (8 μg/kg, Sigma-Aldrich). After 2 hours ad libitum feeding post-injection, food intake was recorded. The anorexia signal by CCK-8 will be transmitted through vagus nerve to acutely decrease satiety.

### Hippocampal Dentate Gyms (DG) and Ammon's Horn Microdissection

Microdissection of the DG and Ammon's horn was performed according to (Hagihara, 2009). Briefly, brains were sampled from deeply anesthetized mice and placed in cold PBS for five minutes. The midbrain, brainstem, and cerebellum were removed so that only cerebrum remained. The cerebrum was sagittally sliced along the midline of the brain. The cerebral hemisphere was placed in ice-cold PBS with the Petri dish and the thalamus and hypothalamus were removed under a dissection microscope. Once the medial side of the hippocampus was exposed, the DG could be readily visualized. A 27-gauge needle was inserted into the edge of DG and moved along the septo-temporal axis of the hippocampus to retrieve the DG. The rest of the hippocampus (Ammon's horn) was collected by sharp forceps. The tissues were pooled from both hemispheres and placed in RNAlater (Qiagen) for storage. The samples were stored at −80° C. until RNA extraction.

### Brain Sampling for IEGs Expression

GF mice were transferred out of isolator and temporally co-housed with stranger GF mice from multiple cages. The brains of GF mice were immediately collected and dissected into hippocampus, hypothalamus, midbrain, and brainstem regions. Brains of SPF mice were sampled and handled following the same procedure. The primers for IEGs were adapted from (Yoshioka, 2012).

RNA Extraction and Quantitative Real-Time Polymerase Chain Reaction (qRT-PCR)

RNA extraction of brain samples was based on the manufacturer's protocol (Rneasy Mini Kit; Qiagen). RNA concentration and quality was measured by NanoDrop (Thermo Scientific). 1 μg RNA from each sample was reverse transcribed using the iScript cDNA synthesis kit (Bio-Rad).

Gene expression of brain subregions was measured using Power SYBR Green PCR master mix (ThermoFisher Scientific) and analyzed using ABI Prism 7900HT system (Life Technologies). Gene expression was normalized to Actb or Gapdh mRNA. Data are presented as fold-change in gene expression in each group relative to control group. The primer sequences were adapted from the Harvard PrimerBank database (Spandidos, 2010).

### Fluorogold Labeling

To label neurons in the PVN in a retrograde manner, mice were given a single intraperitoneal injection of 100 μl Fluorogold (2% w/v; Fluorochrome). The mice were perfused six days post-injection (Wamsteeker Cusulin, 2013). Brains were sampled and stained using the standard protocols described above.

### Retrograde Tracing

The procedure of stereotaxic injection was performed as described above. SPF, GF, vehicle, and ABX mice were stereotaxically injected into the PVN with 46 μl cholera toxin subunit B (CTB)-488 (0.5% w/v; C22841; ThermoFisher). Vehicle and ABX mice were stereotaxically injected into the BNST with 46 μl Fluorogold (2% w/v; Fluorochrome). Stereotaxic injection coordinates (in millimeters): PVN (AP: −0.80, ML: ±0.25; DV: −4.75), BNST (AP: 0.26, ML: ±0.90; DV: −4.30). Mice were perfused one-week post-injection. GF mice were maintained under ABX treatment as described above to limit microbial contamination. All surgically injected animals underwent histological examination to ensure the tracers were correctly injected.

Brain Sample Collection for c-Fos Staining

All brain samples for c-Fos expression were collected one hour after the reciprocal social interaction test. Mice were anesthetized by intraperitoneal injection with a mixture of 100 mg/kg ketamine and 10 mg/kg xylazine. The mice were then perfused via the cardiovascular system with PBS followed by 4% paraformaldehyde (Electron Microscopy Sciences). Brains were removed and post-fixed in 4% paraformaldehyde 3-5 days at 4° C. The brains were kept in PBS with 0.02% sodium azide at 4° C. until sectioning.

### Brain Sectioning and Immunohistochemistry

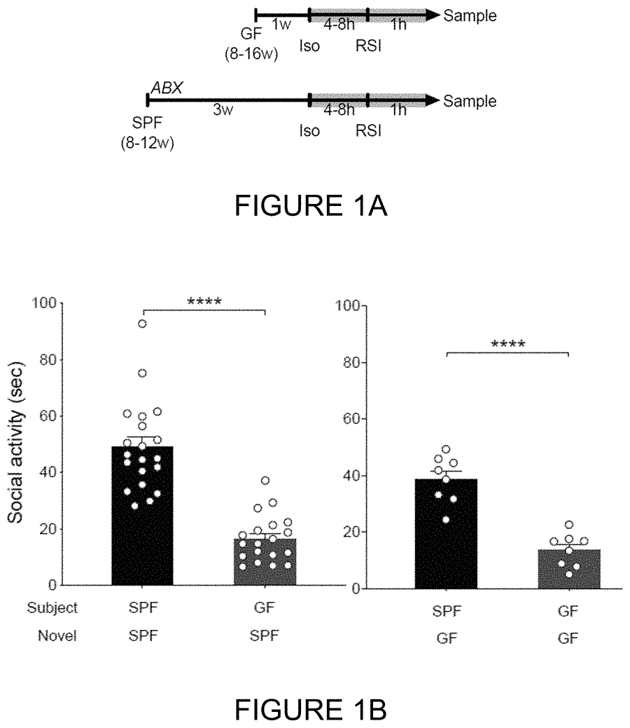

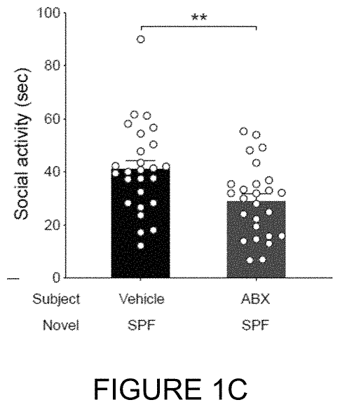

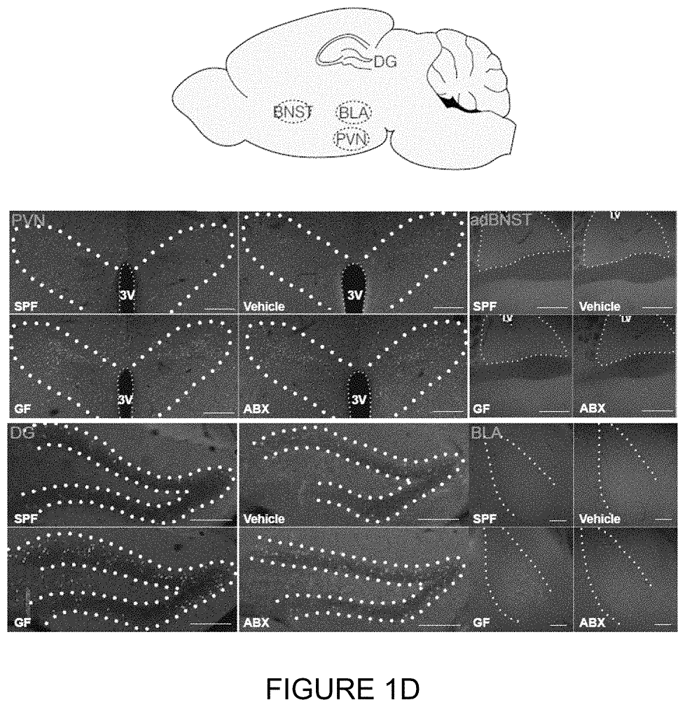

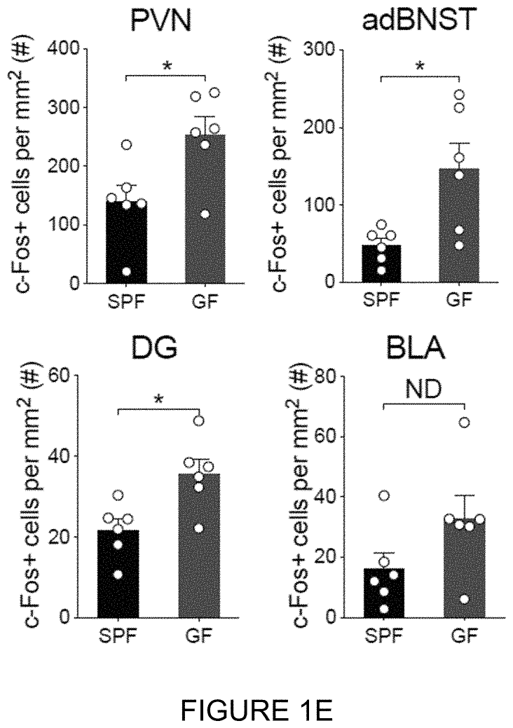

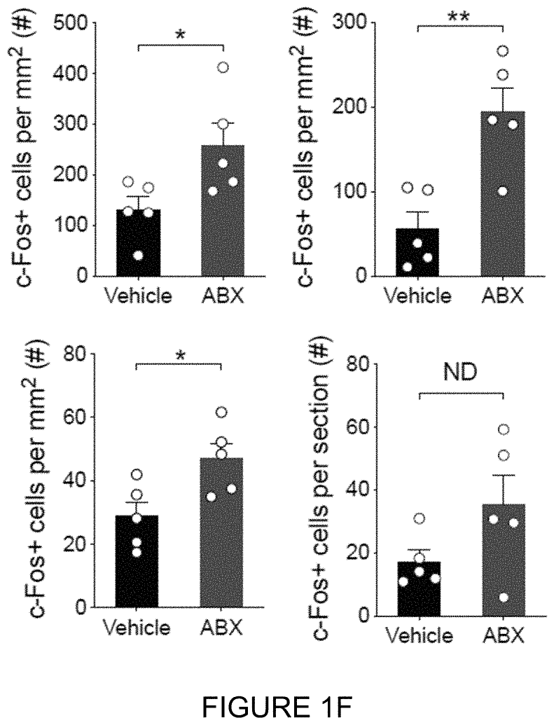

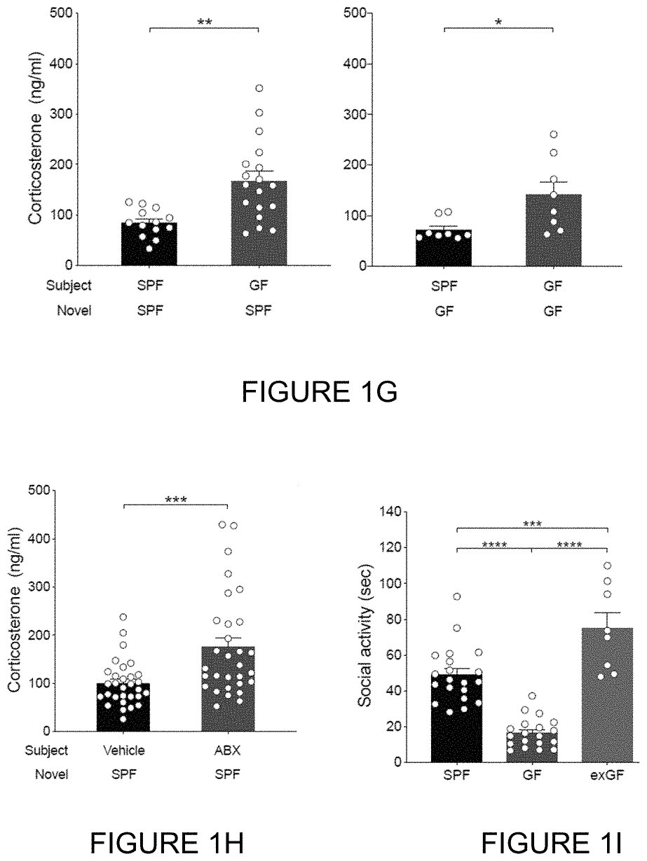

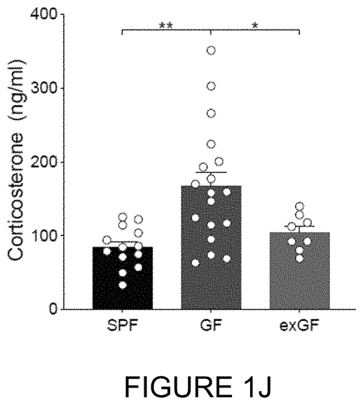

The brains were embedded in 4% UltraPure low melting point agarose (ThermoFisher) and were coronally sectioned by vibratome (VT1000S; Leica) at a thickness of 50 μm, with the exception of the brain sections for FIGS. 1D-1F that were sectioned at a thickness of 100 μm. Brain sections of 50 μm were collected and stained every 0.15 mm. Brain sections of 100 μm were collected sequentially and stained entirely. The brain sections were stored as free-floating in PBS with 0.02% sodium azide at 4° C. until staining. The free-floating sections were incubated with primary antibody in blocking solution (10% horse serum, 0.1% triton X-100, and 0.02% sodium azide in PBS) overnight at room temperature. The next day, sections were incubated with fluorescence-conjugated secondary antibody for 1.5-2 hours at room temperature. Between each step and after secondary antibody staining, sections were thoroughly washed with PBS or PBS with 0.1% triton-X-100 at least three times for 5 minutes each. The stained free-floating sections were then mounted onto the Superfrost Plus microscope slides (Fisher Scientific) in PBS. Excess PBS from adhered sections were carefully removed. Slides were dried at room temperature for 2-5 minutes. 150-200 μl of ProLong Diamond, anti-fade mountant with DAPI (ThermoFisher Scientific) were applied to the slides before placing the coverslip. The slides were immersed with mountant overnight before imaging. Primary antibodies and their dilutions were: goat anti-c-Fos (1:250; SC-52; Santa Cruz), mouse anti-NeuN (1:1000; MAB377; Millpore Sigma), rabbit anti-oxytocin (1:10,000; 20068; Immunostar), rabbit anti-vasopressin (1:2000; 20069; Immunostar), and rabbit anti-Fluorescent Gold (1:1000; AB153-I; EMD Millpore Sigma). The fluorescence-conjugated secondary antibodies were donkey anti-goat (1:1000), donkey anti-rabbit (1:1000), and donkey anti-mouse (1:1000) (ThermoFisher Scientific).

### Microscopic Imaging and Image Analysis

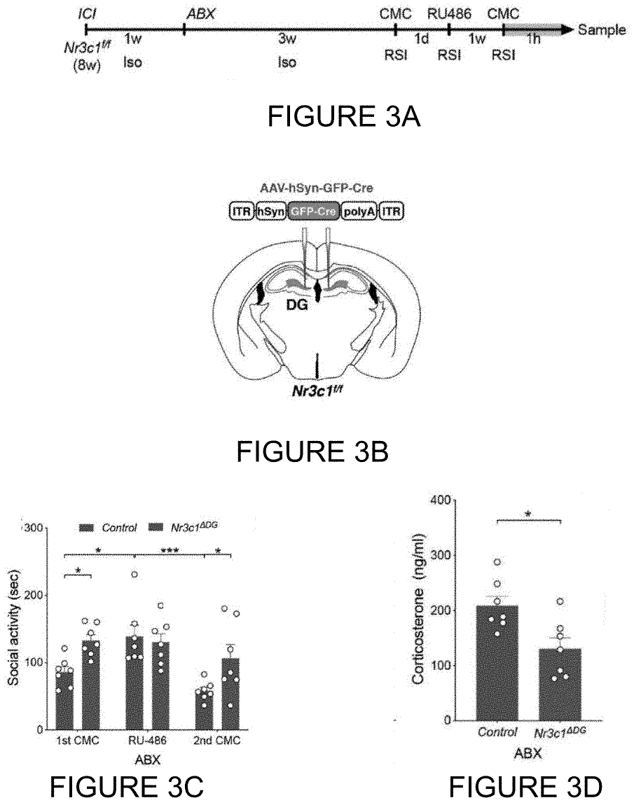

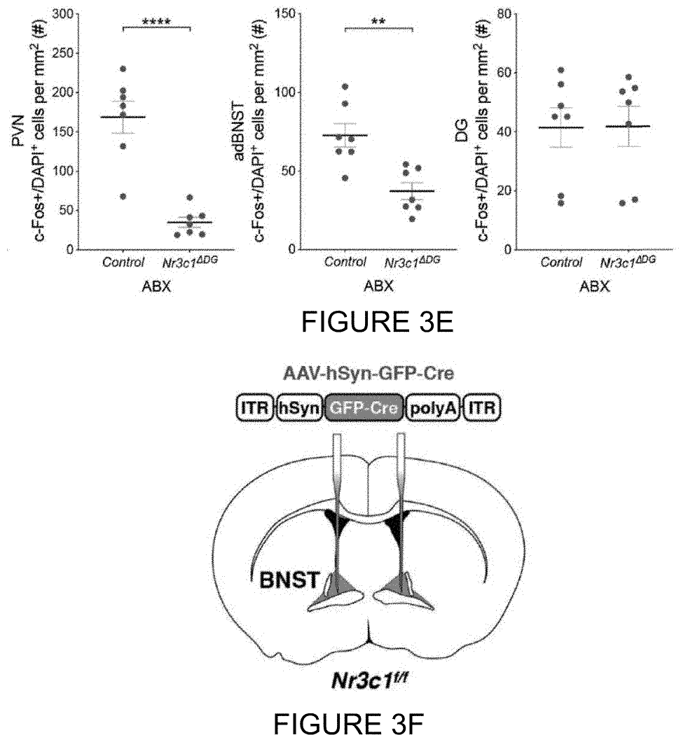

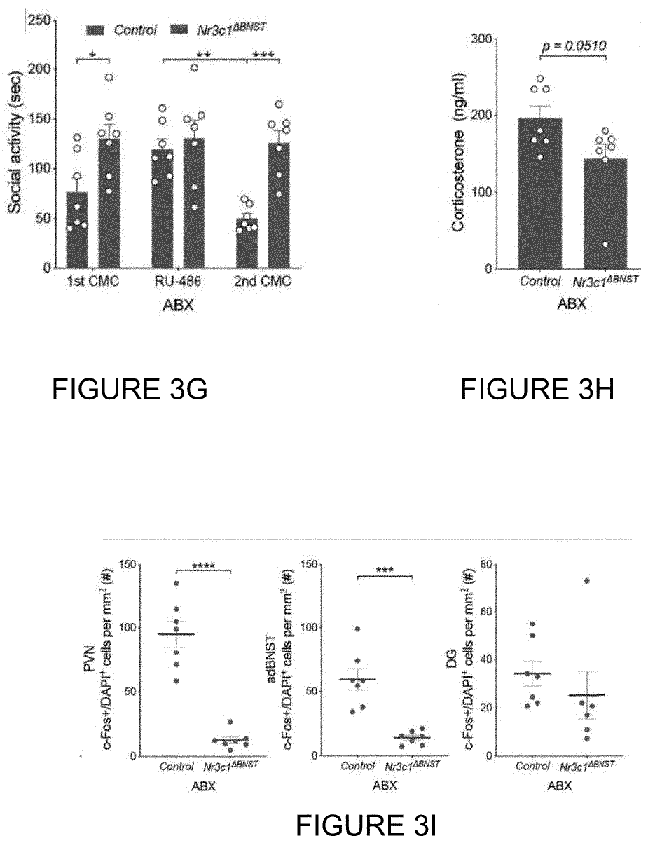

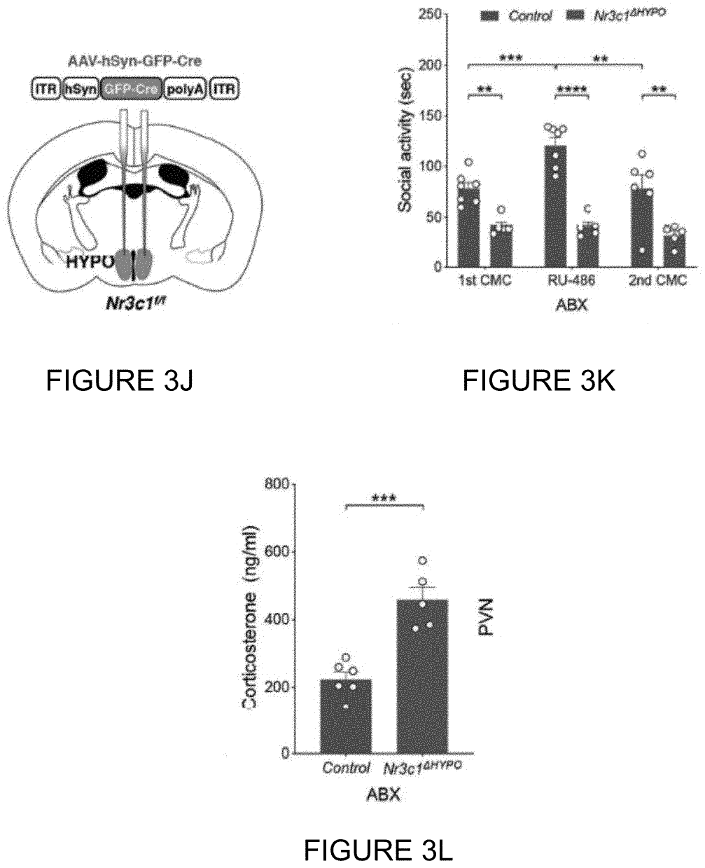

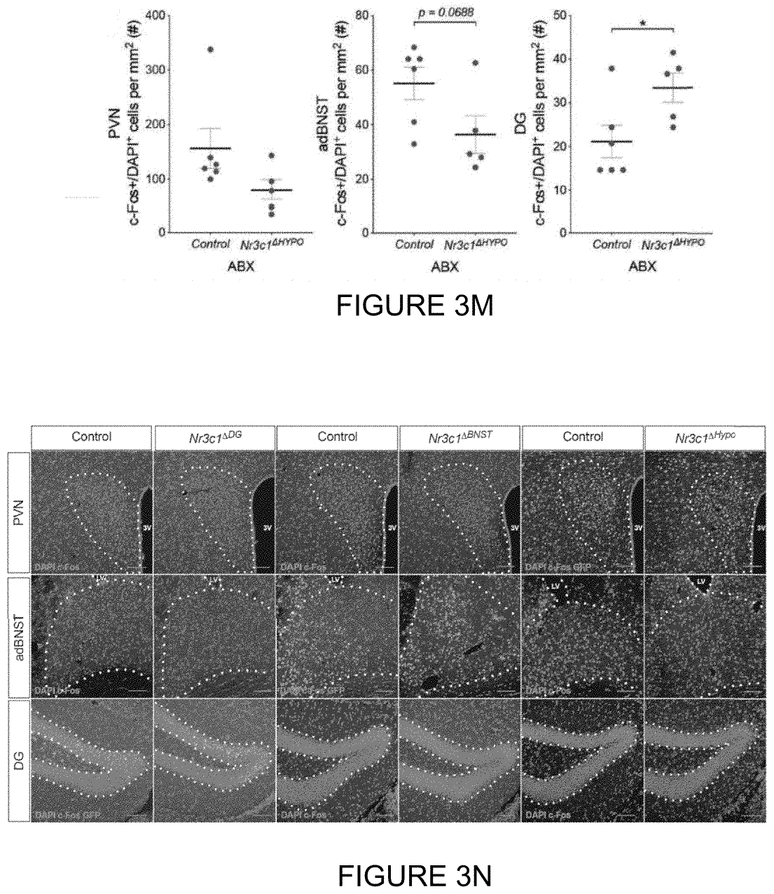

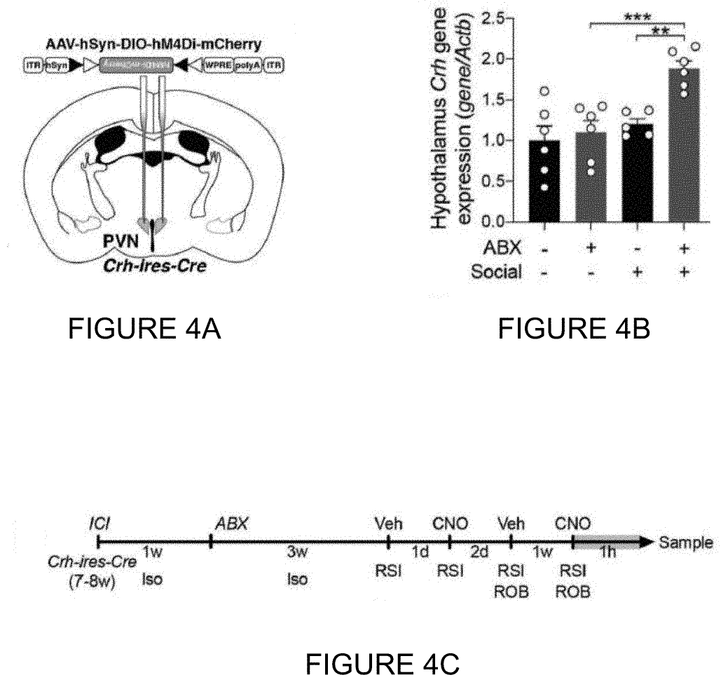

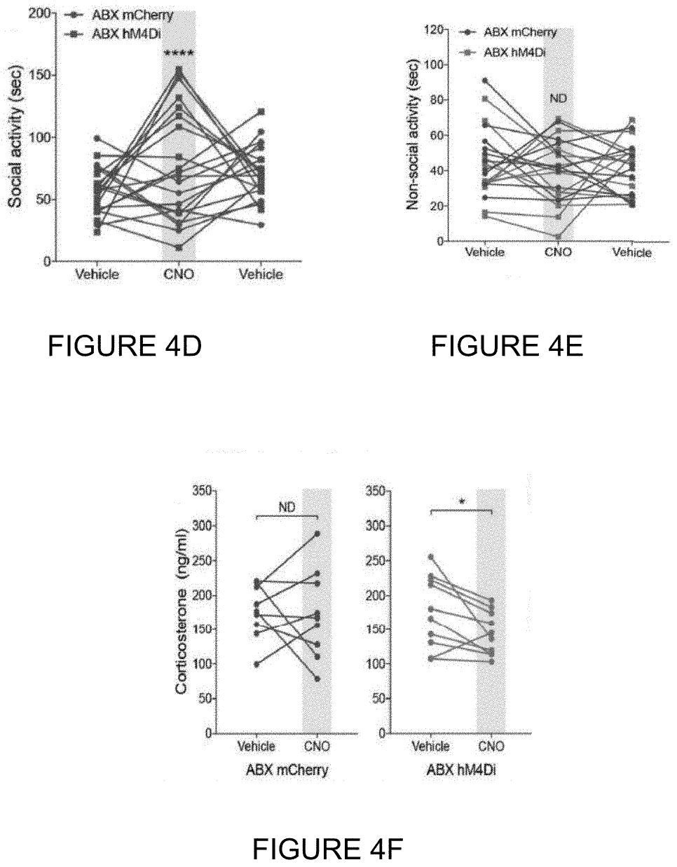

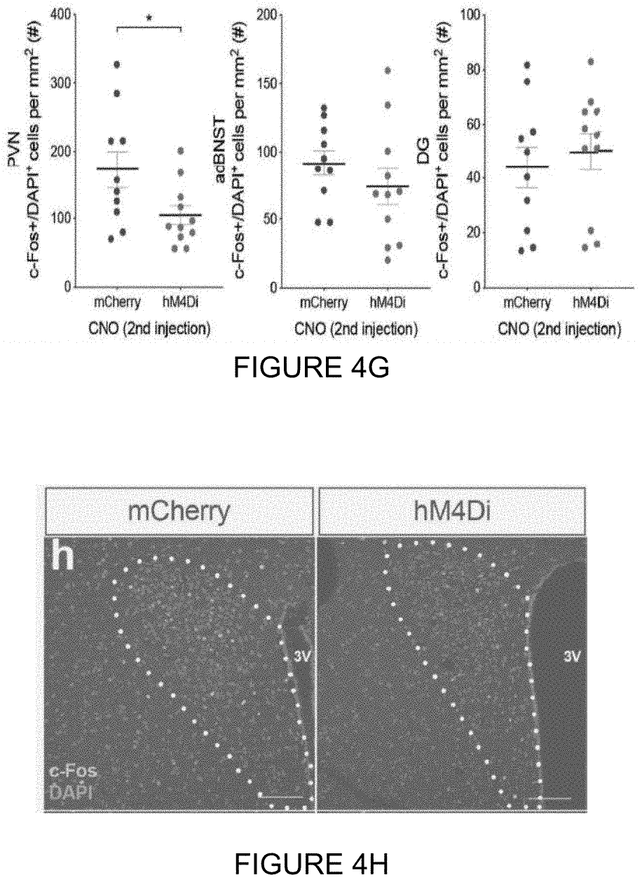

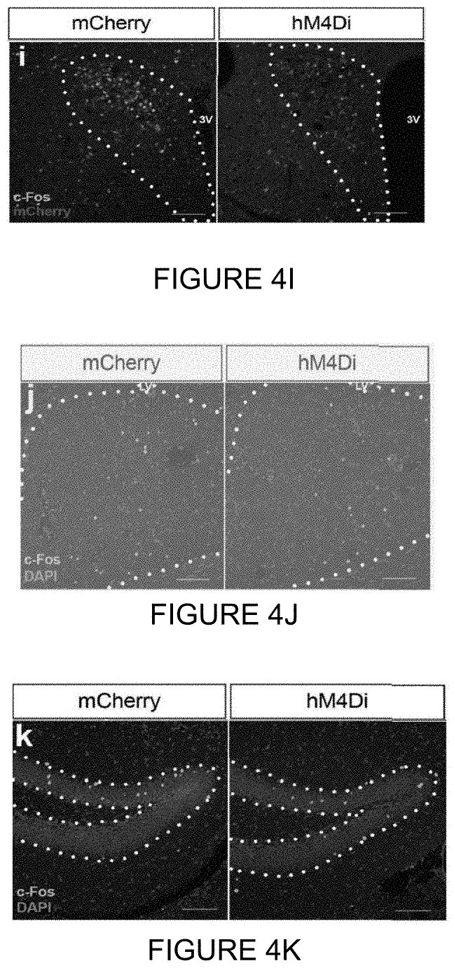

Imaging was performed using the Zeiss LSM 800 inverted confocal laser scanning microscope (Carl Zeiss) with Zen software (Carl Zeiss). For FIG. 1D, and the DG in FIG. 3E, FIG. 3I, FIG. 3M, FIG. 3N and FIGS. 4H-4K, confocal images were obtained by Z-stacks covering the entire Z-axis range of the sections. The interval for each focal plane was 2 μm intervals. The images were then projected in the visualization plane with maximum intensity voxels by maximum intensity projection using Zen software. In addition, the DG and PVN for FIG. 1D were imaged in tiled images covering the entire brain area using Zen software. For other images shown in this study, confocal images were captured in single plane with the highest intensity of DAPI. 20× objective lens was used for all images, except those in FIG. 1D-1F, which were taken using a 10× objective lens. c-Fos-, oxytocin-, vasopressin-, CTB-488-, and Fluorogold-positive cells were quantified using a manual cell counter in ImageJ software (NIH). All images were minimally processed with brightness and contrast adjustment. The adjustment was applied equally across the entire image and consistent in the corresponding controls. Regions of interest were selected by a segmented line based on the anatomical features of each region. The final number of positive cells reported is averaged from multiple images.

Coordinates for adBNST imaging were from anterior to posterior (AP) +0.38 mm to +0.26 mm, (DV) 4.00 mm to +4.50 mm relative to Bregma (bilateral). Coordinates for PVN imaging were from anterior to posterior (AP) −0.70 mm to −0.94 mm relative to Bregma (bilateral). Coordinates for DG imaging were from anterior to posterior (AP) −1.58 mm to −1.94 mm relative to Bregma (bilateral). Coordinates for BLA imaging were from anterior to posterior (AP) −1.06 mm to −2.06 mm relative to Bregma (bilateral). Coordinates for LS imaging were from anterior to posterior (AP) +0.98 mm to +0.86 mm relative to Bregma (bilateral). Coordinates for MeA imaging were from anterior to posterior (AP) −1.46 mm to −1.94 mm relative to Bregma (bilateral).

### Quantification of Fluorogold-Positive Cells in PVN and ME

Confocal images were converted from czi format to jpeg format, and uploaded to ImageJ. Images were smoothed and edges were accentuated to sharpen cellular shape. Brightness and contrast were adjusted to enhance contrast and reduce non-cellular background fluorescence. Images were made binary (16-bit), yielding black and white images in which cells, previously defined by their edges, were white and the rest of the image was black. Images were subjected to a threshold to separate cells from background. The threshold was decided to be that at which cells appeared either partially or completely red when the image was run through the threshold function in the program. Application of this threshold yielded cells completely separate from the background.

The Wastershed, Despeckle, and Fill Holes functions were applied selectively and as needed to divide cells that may have been merged together following binarization, to reduce false positive cells by removing minuscule specks that met threshold, and to close gaps of white space within cells, respectively. The following measurements were programmed to be calculated: “Area”, “Min and Max Gray Value”, “Integrated Density”, and “Mean Gray Value.”

For the PVN: Polygon line tool was used to outline regions of interest. Original confocal images were referenced to ensure accurate outlining. Particles were quantified by an automatic counter that enumerated cells within the outline. Results were given as an image with numbered cells in the region of interest and a table displaying quantification of that image (e.g. cell count, total area, average size of cell, % area that cells take up, mean grey value, and integrated density).

Cell count value was used in the quantification of PVN images. For the ME: Rectangle tool was used to outline a square of known area within an arbitrary part of the median eminence. The same area was applied to other images of the ME at approximately the same part of the ME. Integrated density was calculated for that area. Integrated density was used as a proxy for measuring fluorescence intensity.

### Fecal Microbiota Transplant (FMT)

GF recipient mice were colonized with gut bacteria from SPF donors (exGF) at age of 4 weeks and from ABX-treated donors (AVM, AVNM) at age of 8 weeks. The social behavior test was performed at the age of 10-15 weeks. The fecal samples collected were immediately homogenized in pre-reduced sterilize PBS. 100 μl of the settled suspension was given by oral gavage to GF recipient mice.

### Mouse Fecal Sample Collection and Microbial DNA Extraction

Frozen mouse fecal samples stored in −80° C. until DNA extraction at Laragen Inc (Culver City, Calif., USA). 16S sequencing was performed at Laragen Inc. (Culver City, Calif.), using an in-house validated protocol. Briefly, DNA was extracted using a bead-based method with a proprietary extraction buffer validated against mock community controls. Samples were lysed using Tissuelyser II (Qiagen, Carlsbad, Calif.) and DNA was purified by magnetic beads using the Kingfisher Flex System (ThermoFisher Scientific). Subsequently, 16S sequencing was performed according to the Earth Microbiome Project 16S V4 protocol.

16S rRNA Gene Sequencing and Data Analysis

Demultiplexed sequencing outputs from 2×150 bp V4 16S rRNA gene sequencing using Illumina MiSeq were obtained from Laragen, Inc (Culver City, Calif., USA) and analyzed using the QIIME2 software package (Bolyen, 2019). 55290.5±2427.52 (mean±S.E.M) reads were obtained for vehicle treated samples, 8218.1±5508.82 (mean±S.E.M) reads for AVNM treated samples, and 2045.8±660.83 (mean±S.E.M) reads for negative controls. Paired reads were joined by q2-VSEARCH (Rognes, 2016). Subsequently, reads were quality filtered and denoised using q2-Deblur (Amir, 2017) with left_trim_len=20, trim_length=229 to obtain an operational taxonomic unit (OTU) table and representative sequences. Subsequently, a tree was generated using phylogenetic placement of sequences with SEPP into the GreenGenes reference database (McDonald, 2012) using q2-fragment-insertion (Janssen, 2018).

Taxonomic assignment for each sOTU was obtained by a Naive-Bayes classifier as implemented in q2-feature-classifier (Bokulich, 2018). Lastly, Alpha (observed species and Faith's phylogenetic diversity) and Beta diversity (UniFrac (Lozupone, 2005) metrics were calculated using QIIME2. Hypothesis testing for differences in Alpha diversity were performed by Kruskal-Wallis, differences for Beta diversity were tested by PERMANOVA, as implemented in QIIME2. Raw data were deposited in ENA under BioProject PRJNA632893.

### Isolation and Characterization of Enterococcal Candidate Bacterium

Fecal pellets were taken from mice that received fecal microbiota transplant from AVNM or AVM mice. Pellets were maintained on ice for the duration of sample collection (approximately 20 minutes). Under anaerobic conditions, pellets from each group were then pooled and weighed. Pre-reduced PBS with 1.5% sodium bicarbonate was added at 10 μl PBS+NaHCO₃ per 1mg feces. Feces were mashed and fecal contents completely resuspended using a sterile P1000 pipette tip, vortexed vigorously, and allowed to settle for 1-2 minutes. Supernatant was used to inoculate chopped meat medium (Hardy Diagnostics; AG21H) and incubated anaerobically at 37° C. for five days. Growth differences between groups (e.g. turbidity) were evident in the cultures. 100 μl of each meat broth culture was streaked onto Brucella blood agar plates (Teknova) and incubated anaerobically for three days at 37° C., after which noticeable morphological differences were evident. Single colonies were selected, re-streaked to confirm purity, and colony PCR and sequencing was performed for each isolate (Laragen; Culver City, Calif.) using universal 16S primers: 16s forward 5′-AGAGTTTGATCMTGGCTCAG-3′ (where M is A or C; SEQ ID NO: 1), reverse 5′-GGTTACCTTGTTACGACTT-3′ (SEQ ID NO: 2). Glycerol stocks (30% glycerol) of isolates were stored at −80° C.

Adult Colonization of ABX Mice with Enterococcus faecalis

Gut microbiota were depleted in adult C57BL/6J male mice (8-12 weeks) by treatment with ABX for 3 weeks as described above. Three weeks after ABX treatment, mice were given 6 inoculums of 1×10⁸ CFU each of Enterococcus faecalis (ATCC 19433) by oral gavage (2× per week). ABX water was replaced by sterilized regular drinking water during the period of gavage with Enterococcus faecalis. Control ABX-treated mice and vehicle-treated mice were given 6 oral gavages with 1.5% sodium bicarbonate in PBS (control buffer), which was the same buffer used for E. faecalis gavage. The reciprocal social interaction tested was performed before (1st) and after gavage with E. faecalis or control buffer (2nd) as shown in the timeline scheme (FIG. 6H). The subject mouse was placed in a novel sterilized cage without beddings for a five-minute acclimation period. After the second social interaction test, mice were left in the testing cage for one hour before blood collection. Cardiac puncture blood collection was performed one hour after social interaction. Bacterial genomic DNA from fecal samples were isolated using a commercial kit (Quick-DNA fecal microbe kits, Zymo Research) following the manufacturer's instructions and the relative densities of bacteria were determined by qPCR using Enterococcus faecalis-specific primers—forward 5′-CCCTTATTGTTAGTTGCCATCATT-3′ (SEQ ID NO: 3), reverse 5′-ACTCGTTGTACTTCCCATTGT-3′ (SEQ ID NO: 4).

Perinatal Colonization of Mice with Enterococcal Isolate

3-week old C57BL/6J germ-free male and female mice were mono-colonized with our Enterococcal isolate and setup as breeding pairs. Colonization was confirmed by plating feces on Brucella blood agar plates (Teknova) and PCR of fecal DNA using 16S primers. Offspring from these colonized animals were reared and maintained in a gnotobiotic isolator into adulthood. At 10-11 weeks of age, male offspring were transferred out of the isolator and single-housed in standard, autoclaved cages (Lab Products; Seaford, Del., USA) for 1 week. After 1-week, social behavior was tested and tissues (brain, serum, feces) were collected using methods described above.

### Fecal DNA Extraction for Digital PCR Anchoring for AVM Donors and Recipients

DNA was extracted from all samples by following the Qiagen DNeasy Powersoil Pro Kit protocol (Qiagen; Valencia, Calif., USA). Bead-beating was performed with a FastPrep-24 (MP Biomedicals, Irvine, Calif., USA) Instrument for 1 min at 6.5 m/s. To ensure extraction columns were not overloaded, 30-50 mg of stool as input were used. The elution volume was 100 μl. Stool from germ-free mice and extraction blanks were included to assist in the interpretation of low-abundance samples from antibiotic-treated mice. All samples were stored at −80° C. prior to downstream processing.

Absolute Abundance of 16S rRNA for AVM Donors and Recipients

Absolute taxon abundances were calculated as described previously (Bogatyrev, 2020; Bogatyrev, 2020; Barlow, 2020). Briefly, the total 16S rRNA gene copies per sample was measured using the Bio-Rad QX200 droplet dPCR system (Bio-Rad Laboratories, Hercules, Calif., USA). The dPCR mix contained the following: 1× EvaGreen Droplet Generation Mix (Bio-Rad), 500 nM forward primer and 500 nM reverse primer (Bogatyrev, 2020). Universal primers were modified from the standard 515F-806R primers.

Thermocycling was performed as follows: 95° C. for 5 min, 40 cycles of 95° C. for 30 s, 52° C. for 30 s, and 68° C. for 30 s, with a dye stabilization step of 4° C. for 5 min and 90° C. for 5 min. All ramp rates were 2° C. per second. Enterococcus-specific primers (Balamurugan, 2008) followed similar thermocycling conditions, except the annealing temperature was set to 60° C.

Concentrations of 16S rRNA gene per microliter of extraction were corrected for elution volume and losses during extraction before normalizing to the input sample mass (Eq. 1 (Barlow, 2020).

Microbial Load=dPCR concentration*elution volume*(dead volume)/(extraction volume)*1/(sample mass) (Eq. 1)

Absolute abundance measurements of individual taxa were calculated either by dPCR with taxon-specific primers or by multiplying the total microbial load from Eq. 1 by the relative abundance from 16S rRNA gene amplicon sequencing. The lower limit of quantification (LLOQ) was defined by back-calculating (using Eq. 1) the microbial load from a dPCR reading of 5 copies/μl and assuming a sample mass equal to the average sample mass input.

16S rRNA Gene Amplicon Sequencing for Absolute Abundances for AVM Donors and Recipients

Amplicon libraries were generated as described previously (Bogatyrev, 2020; Bogatyrev, 2020; Barlow, 2020). The variable 4 (V4) region of the 16S rRNA gene was amplified in duplicate with the following PCR reaction components: 1×5 Prime Hotstart mastermix, 1× Evagreen, 500 nM forward primer, and 500 nM reverse primer. Amplification was monitored in a CFX96 RT-PCR machine (Bio-Rad) and samples were removed once fluorescence measurements reached −10,000 RFU (late exponential phase).

Cycling conditions were as follows: 94° C. for 3 min, up to 45 cycles of 94° C. for 45 s, 54° C. for 60 s, and 72° C. for 90 s. Duplicate reactions that amplified were pooled together and quantified with Kapa library quantification kit (Kapa Biosystems, KK4824, Wilmington, Mass., USA) before equimolar sample mixing. Libraries were concentrated and cleaned using AMPureXP beads (Beckman Coulter, Brea, Calif., USA). The final library was quantified using a High Sensitivity D1000 Tapestation (Agilent, Santa Clara, Calif., USA) chip. Sequencing was performed by Fulgent Genetics (Temple City, Calif., USA) using the Illumina MiSeq platform and 2×300 bp reagent kit for paired-end sequencing.

16S rRNA Gene Amplicon Data Processing for AVM Donors and Recipients

Processing of all sequencing data was performed using QIIME 2 2019.1 (Bolyen, 2019). Raw sequence data were demultiplexed and quality filtered using the q2-demux plugin followed by denoising with DADA2 (Callahan, 2016). All samples were rarefied to the read depth of the sample with the minimum number of reads. Taxonomy was assigned to amplicon sequence variants (ASVs) using the q2-feature-classifier (Bokulich, 2018) classify-sklearn naive Bayes taxonomy classifier against the Silva (Quast, 2013) 132 99% OTUs references from the 515F/806R region. All datasets were collapsed to the genus level before downstream analyses. All downstream analyses were performed in IPython primarily through use of the Pandas, Numpy, Seaborn and Scikit-learn libraries. Two independent cohorts of AVM and AVNM donors and recipients were conducted in this study. Initially, Enterococcaceae family was presented in only one cohort of AVM-treated donors (FIG. 6F) but not found in another cohort of AVM donors. Strikingly, Enterococcus genus was presented in both set of AVM recipients. Therefore, the two cohorts of data from AVM recipients were compiled to generate FIG. 6G.

### Behavior Testing

All behavior tests were performed at 8-16 weeks of age. Only male, sexually naive mice were used in this study to avoid the confounding effect of estrous cycle. All behavior tests were conducted in standard sterilized cages. Cage bedding was not changed three days prior to behavioral testing. Mice were acclimated to the testing room for at least 30 minutes before all behavior tests.

Reciprocal social interaction is a widely-used task that tests the social activity toward another novel animal. Two strategies were adopted in this study. For the experiments not involved in survival surgery, mice were group housed and isolated for 4-8 hours prior to testing. The subject mouse was placed in a novel sterilized cage with beddings for a two-minute acclimation period. The same-sex, same strain, sexually naive, novel (stranger) mouse of similar age was introduced into the cage. All interaction between the two mice were video recorded for five minutes. For the experiments involving survival surgery, all mice were single-housed in accordance with IACUC protocols. On the day of behavior testing, the novel mouse of similar age was introduced to the subject's cage. All interaction between the two mice were video recorded for five minutes.

Minimal aggressive behaviors were observed. Each novel mouse was used for testing up to two times per day. Subject mice undergoing repeated testing throughout the experiment were confronted with a novel mouse each time. The following social investigation behaviors constituted “social activity” and were scored as such: anogenital sniffing, nose-nose sniffing, active approach, and push under/crawl. The following solitary behaviors during social interaction constituted “non-social activity” and were scored as such: self-grooming, digging, and rearing. All behaviors were analyzed using of ETHOM software (Shih, 2000).

Three-chamber social test was performed in a 40 (width)×60 (length)×20 (height) cm Plexiglas box divided equally into 3-chambers by transparent walls made by Plexiglas with the opening doors (10 cm width×7 cm height). The procedure consists of two consecutive phases-habituation and sociability. In the habituation phase, test mice were placed in the center of the social chamber for 10 min and allowed to freely explore each compartment. In the sociability phase, test mice were enclosed in the center compartment of social chamber with the doors closed. Two inverted steel wire cups were placed in each of the two side chambers. An unfamiliar, strain-, age- and gender-matched mouse was placed in one of the inverted wire cups. The other inverted wire cup represented as a novel object. After setting up, doors were opened simultaneously, and the test mouse was allowed to investigate the chamber for 10 min. The behavior was recorded by a video camera mounted above the apparatus. Ethovision (Noldus Information Technology) was used to analyze the duration of the mouse in each chamber, frequency entering each chamber, and distance traveled in each phase.

Novel cage behavior is a widely-used test for measuring the non-social activity in a novel environment. The subject mouse was placed in an unused, sterilized cage. Non-social activity was video recorded for 5 minutes. The following solitary behaviors during social interaction constituted “non-social activity” and were scored as such: self-grooming, digging, and rearing. All behaviors were analyzed using of ETHOM software (Shih, 2000).

Open-field test is a widely-used task that tests anxiety and general locomotion. The open-field apparatus is a square open arena (50×50 cm) bordered by opaque plastic walls. Each mouse was placed in along in the interior wall of the arena and behavior was recorded for 60 minutes. The center zone (17×17 cm) was defined as the middle of the open-field chamber. The behavior in the open field was recorded by a video camera mounted over the arena. Ethovision (Noldus Information Technology) was used to analyze the number of entries to, and the duration in, the center zone. Open-field chambers were cleaned with 70% ethanol followed by Rescue disinfectant (Virox Technologies, Inc.) between subjects.

Olfactory habituation/dishabituation is a widely-used test assessing a subject mouse's ability to discriminate a novel odor from a familiar odor after repeated exposure. The mouse is repeatedly presented with the same odor and habituates to it, and distinguishes it from a novel odor that the animal has never been exposed to previously. A subject mouse was acclimated to a sterilized cotton applicator in a new sterilized cage without beddings during the habituation to the testing room. The cotton applicator was inserted into the cage grid and fixed by a weigh dish. The cage lid was intact during the test. Odors were presented in the following order: water, almond extract (McCormick), banana extract (McCormick), C57BL/6J cage odor and, BTBR cage odor (002282; Jackson Laboratory). The neutral (water) and non-social odors (almond, banana) were obtained by dipping the tip of cotton applicator into the solution. The social odors (C57BL/6J cage, BTBR cage) were obtained by swiping the used cage bottom in a zigzag fashion several times. Each odor was presented in triplicate for two minutes per trial. The inter-trial interval was one minute. After each trial, the used cotton applicators were placed in a closed container to prevent cross-trial contamination. The observer was seated a distance away from the testing area to prevent disturbing the mouse. The olfactory investigation behavior was defined as the mouse orienting towards the cotton tip with its nose close to the tip. The behavior was scored using a stopwatch to record the total time spent sniffing.

### Statistical Analysis

All data are represented as mean±standard error mean (SEM). A two-tailed unpaired t-test was used to compare data between two independent groups. A two-tailed paired t-test was used to compare data from the same animal before and after treatment. A one-tailed paired t-test was used to compare data from the same animal staying in mouse chamber and object chamber for 3-chamber social test. Data with more than two independent groups were analyzed by one-way ANOVA with Bonferroni's multiple comparison post-hoc test. Data with two factors were analyzed by two-way ANOVA with Bonferroni's multiple comparison post-hoc test. All data was analyzed using Prism (Graphpad). A p-value was used to justify the significance between groups. When p is smaller than 0.05, the groups are considered as different. The number of biological replicates and the statistical methods were annotated in the figure legends. The number of asterisks indicates the difference in the figures.

### Example 1: The Gut Microbiome Regulates Social Behavior by Activating Stress-Related Brain Regions and Serum Corticosterone Levels

Germ-free (GF) mice display social deficits when encountering a novel, conspecific mouse (Desbonnet, 2014; Buffington, 2016). To explore the neural mechanism(s) for sociability modulated by the microbiota, social behavior was tested using a reciprocal social interaction (RSI) paradigm (FIG. 1A). While social profiles of GF and ABX-treated mice have already established (Desbonnet, 2014), it was consistently observed that GF mice, devoid of gut bacteria, exhibited reduced social activity toward novel stranger mice, regardless of the microbial status of the novel animal (FIG. 1B). Importantly, non-social activity (including rearing, self-grooming, and digging) in this experimental paradigm was indistinguishable between GF and specific pathogen free (SPF) mice that harbor a complex microbiota. Since GF mice have certain developmental defects (Desbonnet, 2014; Buffington, 2016), social behavior in adult SPF mice treated orally with a broad-spectrum antibiotic cocktail (ABX) to postnatally deplete their microbiota was tested (FIG. 1A). ABX mice also displayed decreased social activity when encountering a novel SPF mouse (FIG. 1C), consistent with previous reports (Desbonnet, 2014; Buffington, 2016; Desbonnet, 2015). Social activity was not impacted by a novel ABX mouse. Similarly, non-social activity was unchanged by antibiotic treatment. Culture-dependent and culture-independent analysis demonstrated that the microbiota was fully depleted in GF and ABX mice. Injection of antibiotics directly into the brain or chronic systemic (intraperitoneal) injection of antibiotics did not alter social behavior or locomotion, suggesting the effects of antibiotics are not due to direct neurotoxicity. Depletion of the microbiome reduced social activity regardless of animal gender, social isolation time, and age. Effects in the 3-chamber social test was not ob served.

Anxiety-like behavior was lower in GF mice compared to their SPF counterparts, as previously reported (Diaz Heijtz, 2011); however, there was no change following antibiotic treatment of SPF mice. Overall locomotor activity was not affected in GF or ABX groups, suggesting that the observed social deficits are unlikely to be a consequence of anxiety. GF status does not impact total water intake, and no differences in olfaction were detected between groups of mice in response to water or other neutral volatile odors.GF mice displayed heightened olfactory investigatory behavior when exposed to a complex social odor (soiled bedding). However, habituation to the same social odor and dishabituate to a novel social odor was similar between GF and SPF mice.

In view of the foregoing, this example shows that absence of the gut microbiota either during development or adulthood impairs social activity in mice, and that social deficits are likely not a consequence of olfactory dysfunction, anxiety, or altered locomotion in accordance with some embodiments described herein.

### Example 2: The Gut Microbiome Regulates Social Behavior by Activating Stress-Related Brain Regions

To analyze neuronal activation in the brains of mice after a social interaction task, c-Fos labeling was performed. c-Fos expression was increased after a social encounter in GF and ABX mice, compared to SPF mice, in several brain regions associated with stress responses, including the hippocampal dentate gyms (DG), paraventricular nucleus of the hypothalamus (PVN), and BNST (FIG. 1D-1F). A trend toward increased c-Fos staining in the basolateral amygdala (BLA) of GF and ABX mice (FIG. 1D-1F), previously shown to be involved in different social behavior paradigms was observed (Siuda, 2016). Increased brain activation in GF mice is specifically a result of social interaction, as c-Fos labeling was not different between SPF and GF mice exposed to a novel cage. Upregulation of several immediate early genes (IEGs) in the hippocampus and hypothalamus of GF mice following exposure to stranger animals, with minimal IEGs expression changes in the midbrain and brainstem was observed.

In view of the foregoing, this example shows that depletion of the microbiota activates or de-represses activity in distinct brain regions in response to social interaction in accordance with some embodiments described herein.

### Example 3: The Gut Microbiome Regulates Social Behavior by Activating Corticosterone Levels

Gut bacteria modulate postnatal development of the hypothalamus-pituitary-adrenal (HPA) stress response in mice (Sudo, 2004) and affect corticosterone production by the HPA axis in rodents (Sudo, 2004; Clarke, 2013; Neufeld, 2011; Crumeyrolle-Arias, 2014). Accordingly, serum corticosterone levels in GF and ABX mice were more robustly increased after a transient social encounter compared to SPF controls (FIG. 1G, FIG. 1H). Corticosterone was elevated in GF mice exposed to a novel cage, while corticosterone levels were not changed between vehicle and ABX mice exposed to a novel cage. Interestingly, corticosterone concentrations were increased immediately after social encounter and lasted at least one hour after social interaction in ABX mice. Increased levels of corticosterone in GF and ABX mice were not subject to circadian rhythm. To determine whether social deficits and increased corticosterone in GF mice are reversable postnatally, transplant of gut bacteria from SPF donors into GF mice (exGF) corrected social activity and lowered corticosterone compared to animals that remained GF (FIG. 1I, FIG. 1J).

Vasopressin and oxytocin neuropeptide systems in the PVN have been implicated in social behavior (Donaldson, 2008; Hung, 2017). While SPF and GF mice contain similar numbers of vasopressin expressing neurons, GF mice have fewer oxytocin-expressing neurons in the PVN. Moreover, levels of gene expression for oxytocin, vasopressin, and their receptors in the hypothalamus were similar regardless of microbiome status, suggesting gut bacterial effect on social behavior are likely not mediated through vasopressin while an oxytocin-dependent pathway cannot be completely ruled out.

Since the PVN is a key brain region within the hypothalamus and therefore an integral part of the HPA axis, neurons projecting from the PVN to the hypophyseal pituitary portal were labeled in a retrograde manner by peripheral injection of Fluorogold (Wamsteeker Cusulin, 2013). There were no overt changes in the numbers of neurons in the PVN and their projections to the median eminence (ME) in GF mice. Further, neuronal counts in the PVN were determined by Fluorogold injection into a reporter mouse strain (Crh-ices-Cre;Ai14D) that enables visualization of corticotrophin-releasing hormone (CRH)-expressing neuron, critical in the stress response of mice. No neuronal changes in the PVN were observed after ABX treatment.

In view of the foregoing, this example shows that gut microbiota modulates corticosterone levels and increased corticosterone levels in GF and ABX mice following social interaction are not sue to compensatory effects of stress-related gene expression or changes in PVN neuronal density in accordance with some embodiments described herein.

### Example 4: Inhibition of Glucocorticoid Signaling Rescues Social Deficits

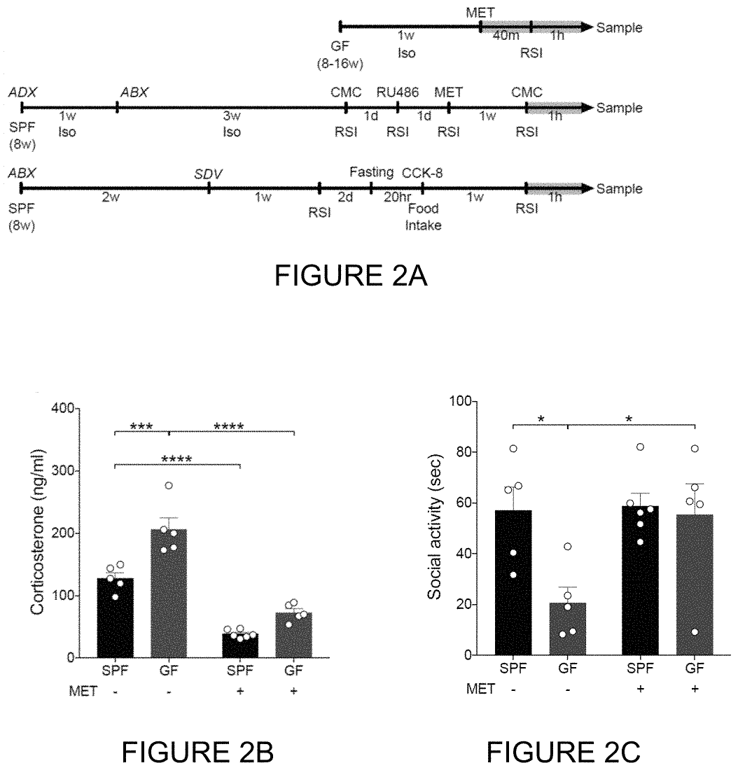

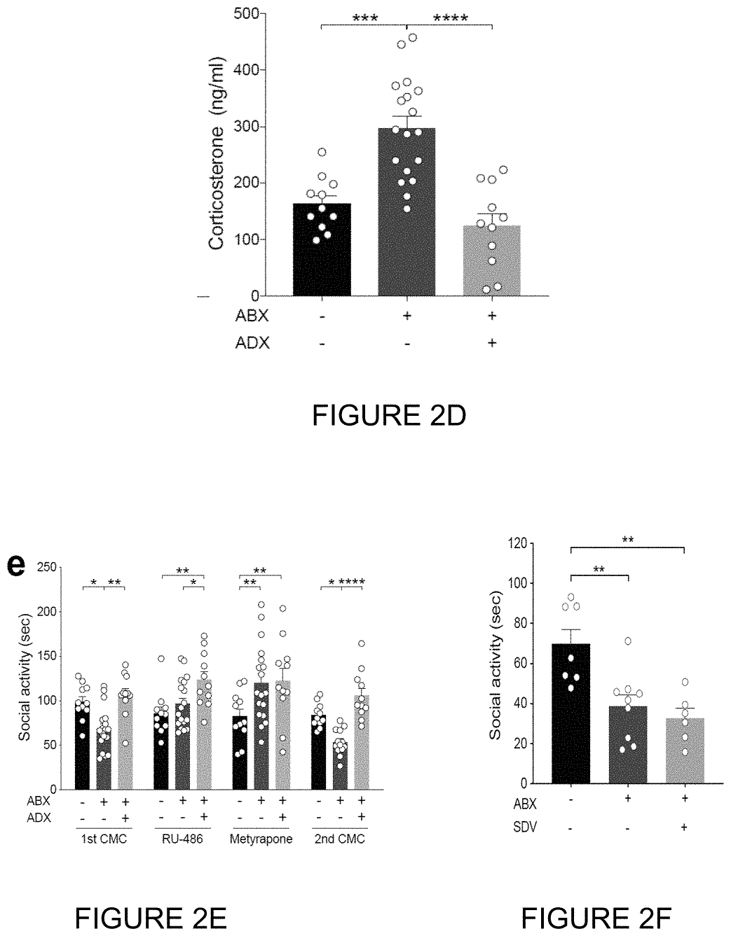

The synthesis and release of corticosterone is predominately controlled by the HPA axis (Bains, 2015). To determine whether corticosterone signaling is required for the social deficits observed in GF and ABX mice, corticosterone production was inhibited using both pharmacological and surgical approaches (FIG. 2A). In GF mice, injection of the corticosterone synthesis blocker, metyrapone (MET), effectively reduced serum corticosterone levels and significantly increased social activity compared to injection of the vehicle control, carboxymethylcellulose (CMC) (FIG. 2B, FIG. 2C). The adrenal gland is a major source for the production of corticosterone (Taves, 2011). As expected, ABX mice displayed increased corticosterone levels compared to vehicle-treated mice after sham surgery (vehicle sham vs ABX sham; FIG. 2D). Adrenalectomy blocked corticosterone increases in ABX mice compared to sham controls (ABX sham vs ABX ADX; FIG. 2D). Importantly, ABX adrenalectomized mice displayed normal social activity (vehicle sham vs ABX ADX; FIG. 2E, 1st CMC and 2nd CMC), while ABX sham mice displayed lowered social activity (vehicle sham vs ABX sham; FIG. 2E, 1st CMC and 2nd CMC) compared to controls. Next, RU-486 was administered to antagonize the glucocorticoid receptor, or MET to inhibit corticosterone synthesis, including to adrenalectomized mice to confirm there is no effect of extra-adrenal glucocorticoids. During RU-486 treatment, social activity was similar between vehicle and ABX sham mice, while MET treatment increased social activity in ABX sham mice compared to vehicle sham mice (FIG. 2E). However, ABX adrenalectomized mice exhibited elevated social activity under both RU-486 and MET treatment compared to ABX sham and vehicle sham mice (FIG. 2E). The effect of drug treatment is reversible, as social activity was similar between the first and second CMC injections (FIG. 2E). On the contrary, acute corticosterone injection impaired social behavior in all groups. Minimal effects on non-social activity by adrenalectomy or drug-induced modulation of corticosterone signaling was observed in the ABX groups.

The vagus nerve is a critical pathway for gut-brain communication (Sgritta, 2019). Glucocorticoid receptors are expressed in the gut and have been shown to affect metabolism in the GI tract (Pressley, 1975; Reichardt, 2012). Therefore, to test whether the vagus nerve could relay microbiome-mediated signals that impact social behavior, subdiaphragmatic vagotomy (SDV) in ABX mice was performed and evaluated social interaction. Social behavior and corticosterone levels were not changed by vagotomy in ABX mice (ABX sham vs ABX SDV; FIG. 2F), suggesting that that vagus nerve does not participate in social impairment following microbiota depletion.

Next, the gene encoding the for glucocorticoid receptor (Nr3cl) in specific brain regions was deleted by stereotaxic injection of AAV-hSyn-Cre-GFP bilaterally into the DG, BNST and hypothalamus of Nr3cl^{f/f} mice, and evaluated social behavior, serum corticosterone levels and c-Fos (FIG. 3A). The hippocampus and BNST are regions involved in upstream signaling to the HPA axis (Bains, 2015). In ABX treated mice, knockout of glucocorticoid receptors in the DG (Nr3cl^{ΔDG}) or BNST (Nr3cl^{ΔBNST}) resulted in increased social activity compared to wild-type mice injected with AAV-hSyn-Cre-GFP and treated with ABX (control group) (FIG. 3B, FIG. 3C, FIG. 3F, FIG. 3G). Pan blockade of glucocorticoid receptor signaling by RU-486 enhanced social activity only in ABX control mice, but not ABX Nr3cl^{ΔDG} or Nr3cl^{ΔBNST} mice, suggesting ablation of this receptor in either brain region is sufficient to rescue social deficits (FIG. 3C, FIG. 3G). Accordingly, serum corticosterone levels (FIG. 3D, FIG. 3H) and c-Fos expression (FIG. 3E, FIG. 3I, FIG. 3N) in the PVN and BNST were reduced in ABX Nr3cl^{ΔDG} and Nr3cl^{ΔBNST} mice after social interaction. Increased social behavior and decreased corticosterone in Nr3cl^{ΔDG} and Nr3cl^{ΔBNST} mice were observed only following antibiotic treatment, but not in mice with an intact microbiome with the exception of social activity in SPF Nr3cl^{ΔBNST} mice.

Bilateral stereotaxic delivery of AAV-hSyn-Cre-GFP into the hypothalamic region of Nr3cl^{f/f} mice (Nr3cl^{ΔHYPO}) decreased social behavior compared to non-ablated control mice following ABX treatment, with or without RU-486 administration (FIG. 3J, FIG. 3K). Serum corticosterone (FIG. 3L) and c-Fos expression in the DG (FIG. 3M, FIG. 3N) were significantly increased in ABX-treated Nr3cl^{ΔHYPO} mice, supporting the notion that the glucocorticoid receptor in the hypothalamus serves as a negative regulator of the HPA axis (Bains, 2015). Importantly, non-social activity was not affected in Nr3cl^{ΔDG} and Nr3cl^{ΔHYPO} mice, but was reduced in ABX-treated Nr3cl^{ΔBNST} animals. Decreased social behavior and increased corticosterone in Nr3cl^{ΔHYPO} mice were observed only following antibiotic treatment. Taken together, glucocorticoid receptors play distinct roles in different brain regions, impacting levels of corticosterone and social behaviors regulated by the microbiota.

### Example 5: Inactivation of CRH Neurons in the PVN Improves Social Behavior

Since pharmacologic suppression and genetic ablation of corticosterone signaling successfully corrected the social impairment observed in ABX mice, CRH-expressing neuron may be involved. While global changes in stress-related genes were not observed in the DG, Ammon's horn, or hypothalamus, the Crh gene was selectively upregulated in the hypothalamus of ABX mice after social interaction, but not following novel cage exposure (FIG. 4A). Next, chemogenetics using ‘designer receptors exclusively activated by designer drugs’ (DREADDs) were used to investigate whether social deficits in ABX mice are a direct result of CRH neuronal activation in the PVN (Urban, 2015). To locally inactivate neurons, a mutated human M4 muscarinic receptor, hM4Di, that induces neuronal silencing (Urban, 2015) was delivered into the hypothalamus of mice. Specifically, AAV-hSyn-DIO-hM4Di-mCherry (hM4Di) was bilaterally injected into the PVN of Crh-ires-Cre mice, using AAV-hSyn-DIO-mCherry (mCherry) as a control (FIG. 4B, FIG. 4C). Chemogenetic inactivation of CRH-expressing neurons in the PVN by intraperitoneal injection of clozapine N-oxide (CNO), a designer drug for DREADDs, dramatically increased social behavior in ABX mice in an acute fashion, whereas no change in social behavior was observed following CNO injection to mice receiving control virus (FIG. 4D). Moreover, injection of CNO decreased corticosterone in ABX hM4Di mice, but not in ABX mCherry mice (FIG. 4F). Inactivation of CRH neurons in the PVN also decreased c-Fos staining after social interaction (FIG. 4G-4I), but interestingly did not alter c-Fos expression in the BNST or DG (FIG. 4G, FIG. 4J, FIG. 4K). Specificity of c-Fos staining to CRH neurons was unable to be determined.

Next, AAV-hSyn-DIO-hM4Di-mCherry was delivered bilaterally into the BNST, another region with high expression of CRH. Remarkably, social behavior, corticosterone levels, and brain c-Fos expression were not affected during silencing of CRH neurons in the BNST . Non-social activity was unchanged following inactivation of CRH neurons in the PVN or BNST (FIG. 4E).

Effects of the microbiota on PVN neurons can result from changes in neural circuitry and/or neural activity. To assess circuitry, the retrograde neural tracer cholera toxin B subunit (CTB) was unilaterally injected into the PVN of SPF and GF mice , and labeled projections from the BNST, lateral septum (LS), and the medial amygdala (MeA) in the ipsilateral hemisphere, but not projections from the hippocampus and BLA. CTB-labeled neuronal projections from the PVN in the BNST, LS and MeA trended lower in SPF vs GF mice, though results were not statistically different. To define neural projection upstream to the PVN, CTB was co-injected into the PVN and Fluorogold into the BNST of ABX Crh-ires-Cre;Ai14D reporter mice. The PVN receives similar projections from the BNST, LS, and MeA in both ABX and control mice. Labeling alternative projections to the BNST, from the PVN, LS, and MeA showed that the PVN and BNST are bidirectionally connected to each other and receive neuronal projections from the LS and MeA. Importantly, similar retrograde labeling from the PVN and BNST was observed in both ABX and vehicle mice. Therefore, decreased social behavior and increased corticosterone levels in GF and ABX mice are likely due to changes in neural activity rather than neural circuitry, with the PVN being the major region impacted by the microbiota.

### Example 6: Activation of Stress Signaling Pathways in the Brain Promote Social Deficits

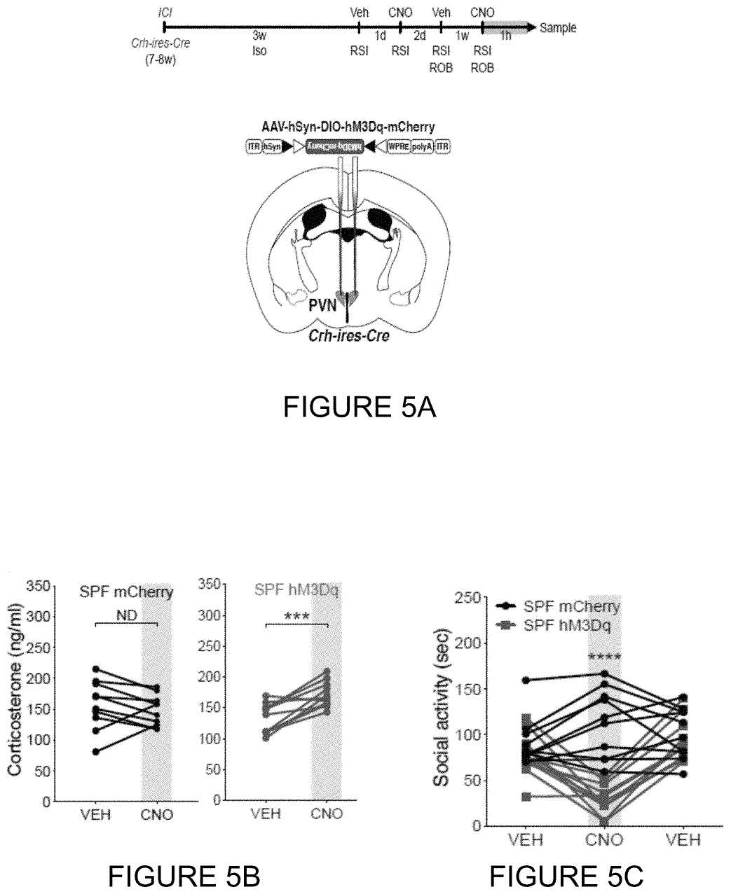

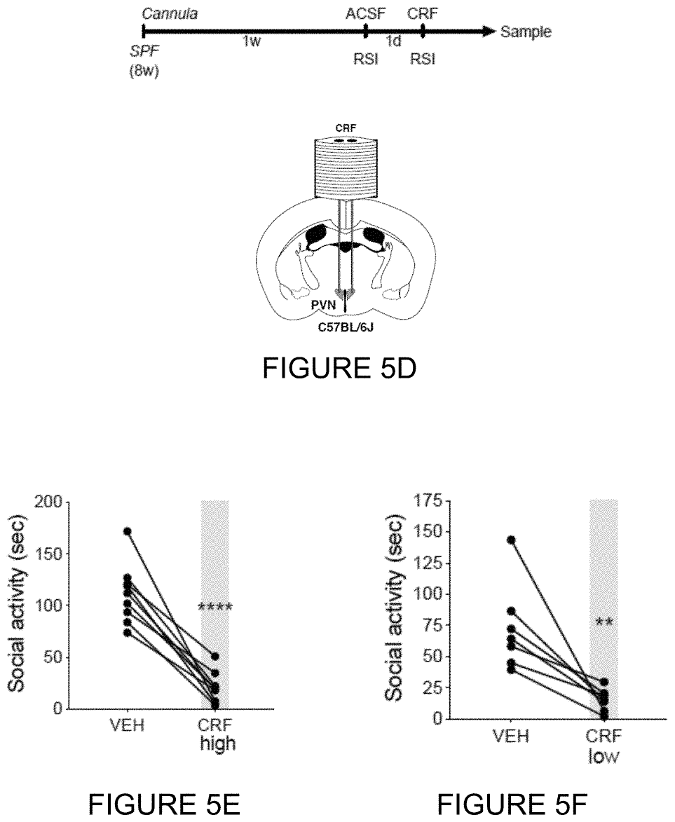

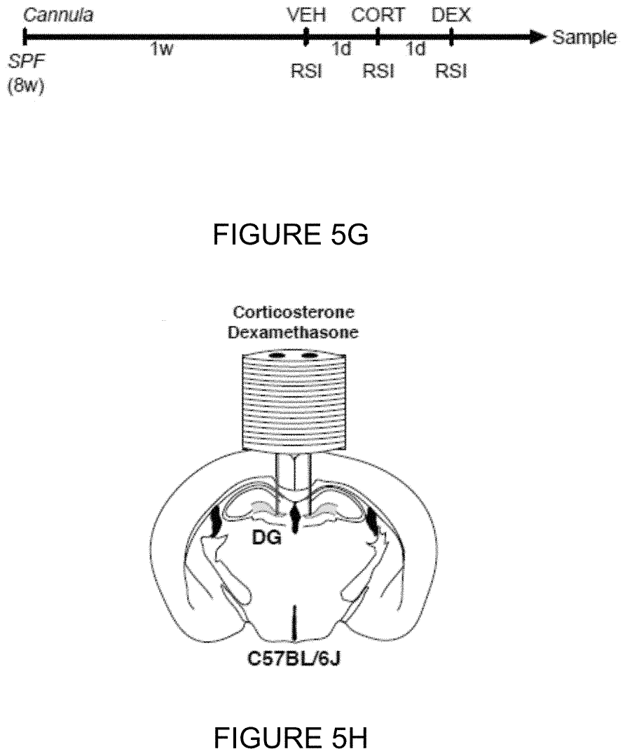

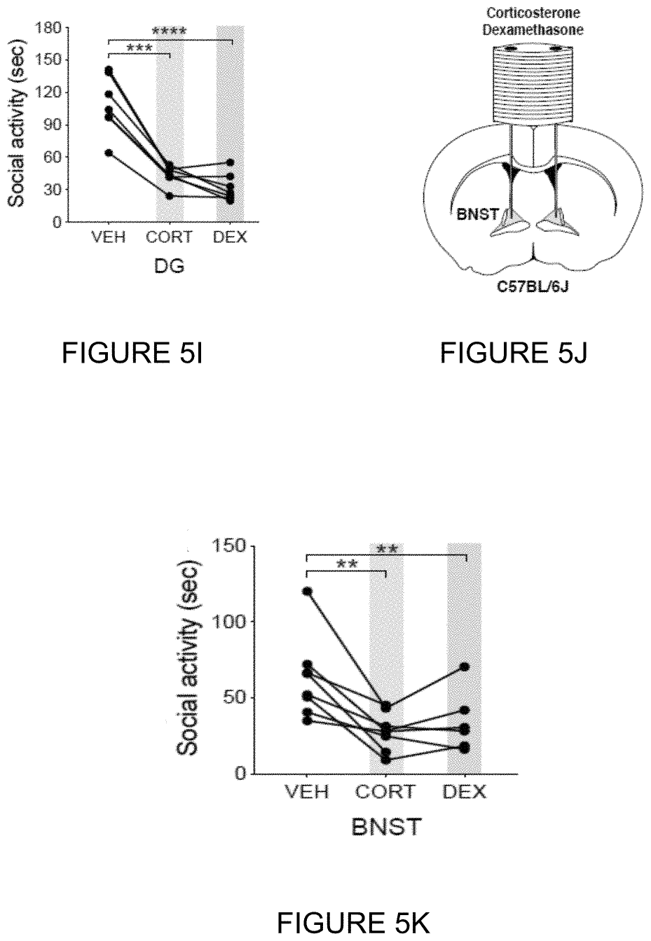

Exploring brain circuits in mice with and without microbiomes led to uncovering of a link between CRH neurons in the PVN and social behavior. To test whether activation of CRH neurons in the PVN is sufficient to induce social impairment under more “natural” conditions, AAV-hSyn-DIO-hM3Dq-mCherry (hM3Dq) was bilaterally injected into the PVN of Crh-ires-Cre mice with an SPF microbiota, using AAV-hSyn-DIO-mCherry (mCherry) injected mice as controls (FIG. 5A). hM3Dq is a mutated human M3 muscarinic receptor that induces neuronal firing when activated (Urban, 2015). Injection of CNO increased corticosterone in SPF hM3Dq mice, but not in SPF mCherry mice (FIG. 5B). Importantly, chemogenetic activation of CRH neurons in the PVN following intraperitoneal injection of CNO significantly decreased social behavior in SPF hM3Dq mice in an acute fashion, but not in SPF mCherry mice (FIG. 5C) or SPF hM3Dq injected with vehicle. Interestingly, chemogenetic activation of PVN CRH neurons also increased non-social activity in SPF hM3Dq mice, which was also observed in mice under stress conditions (Fuzesi, 2016).

Injection of synthetic corticotropin-releasing factor (CRF) into the PVN of SPF mice dramatically reduced social activity toward a novel mouse (FIG. 5D-5F). Interestingly, CRF levels impact non-social activity, where low levels of CRF increased non-social activity and high levels reversed these changes. Furthermore, injection of corticosterone or the glucocorticoid receptor agonist dexamethasone (DEX) into the DG and BNST of SPF mice also decreased social activity (FIG. 5G-5K). Activation of CRH neurons in the PVN and glucocorticoid receptor neurons in the DG and BNST of naive mice are sufficient to induce social alterations, in response to signals from gut bacteria, revealing a neural pathway that regulates social behavior. This specific population of neurons may also mediate social activity to non-microbial cues.

### Example 7: Treatment with E. faecalis Improves Social Behavior

Gut bacterial species that impact social activity in mice were next identified. The four antibiotic cocktail that promotes behavioral deficits targets a broad spectrum of bacteria. Treatment of SPF mice with different combinations of antibiotics followed by behavior testing uncovered that a microbe(s) exclusively sensitive to neomycin appears to be responsible for modulating social activity and c-Fos expression in the PVN (FIG. 6A-6B). Most antibiotic combinations, except that excluding neomycin, reduced social behavior, indicating effects on social activity are not a broadly conserved feature of many gut bacteria.

Accordingly, corticosterone levels were lower after social interaction in AVM-treated mice compared to AVNM treatment (FIG. 6C). Remarkably, transplant of microbiota from antibiotic-treated donor mice into untreated GF recipients transferred the associated social activity phenotypes from AVM and AVNM mice (FIG. 6D), strongly suggesting specific (neomycin sensitive) bacterial species mediate this behavioral effect. Further, decreased serum corticosterone profiles were also transferred from donor to recipient mice in a manner dependent on neomycin (FIG. 6E).

Standard bacterial 16S rRNA gene sequencing methods did not allow measurement of microbial representation and abundance due to low biomass in fecal samples following microbiome depletion with antibiotics. The absolute abundance and identity of microbes retained following transplant of AVM and AVNM microbiota into GF recipients was determined by employing a new quantitative sequencing framework that utilizes the high precision of digital polymerase chain reaction (dPCR) to anchor 16S rRNA gene amplicon sequencing measurements (Bogatyrev, 2020; Bogatyrev, 2020; Barlow, 2020). An Enterococcus genus solely present in AVM-treated and AVM-microbiota recipient mice was identified, which is absent in mice that received fecal samples from AVNM donors (FIG. 6F, FIG. 6G). Via microbiological culturing of feces and direct 16S rRNA sequencing from colonies, the predominant bacterial species from the AVM microbiota as Enterococcus faecalis (E.f.) was identified (FIG. 6G).

To determine whether E. faecalis is able to modulate social behavior and corticosterone levels, adult SPF mice were treated for 3 weeks with ABX as previously, then switched to regular water and colonized with E. faecalis by gavage (ABX+E.f.) or treated with sodium bicarbonate control (ABX+Ctrl) (FIG. 6H). Matched vehicle (VEH) mice (no antibiotics) were also gavaged with buffer. Animals were first behavior tested before gavage (1st trial) to confirm that antibiotics reduced social activity in this paradigm (FIG. 61). Strikingly, following 3 weeks of colonization with E. faecalis, ABX+E.f. mice increased their social activity between the 1st and 2nd trials, whereas VEH+Ctrl and ABX+Ctrl mice showed no change in behavior over this time (FIG. 6J, FIG. 6K; compare 1st to 2nd RSI in FIG. 6J). As expected, corticosterone levels were reduced in ABX+E.f. mice compared to ABX+Ctrl mice to levels similar to vehicle animals (FIG. 6L). These data reveal that E. faecalis increases social activity in mice with ABX-depleted microbiota, but do not exclude other bacteria with similar effects. Further, germ-free mice mono-colonized with E. faecalis, bred and offspring behavior tested also showed an increase in social activity and decreased c-Fos expression in the brain compared to GF mice, with corticosterone levels unaffected potentially due to developmental issues in GF mice or lack of other microbes. Collectively, members of the gut microbiota can modulate the HPA axis to impact social behavior in mice.

### Example 8: Treatment of Social Behavioral Deficit

This example illustrates the treatment of a patient suffering from a behavioral deficit. A human subject who exhibits behaviorally deficit symptoms is identified. The level of corticosterone in the subject is determined. A composition comprising an effective amount of E. faecalis is administered to the subject via oral administration. The administration of E. faecalis is expected to alter the corticosterone level and the composition of gut bacteria in the subject. It is also expected that the bacterial administration will improve behavioral performance in the subject.

### Example 9: Treatment of Social Behavioral Deficit

A human subject who exhibits behaviorally deficit symptoms is identified. The level of corticosterone in the subject is determined. A composition comprising an effective amount of E. faecalis is administered to the subject via oral administration. The administration of E. faecalis is expected to alter the level of a product of a gene of the subject selected from the group consisting of: Crhl (or an ortholog thereof), c-Fos (or an ortholog thereof), Nr3cl (or an ortholog thereof), a human ortholog of any of the listed genes, or a combination of two or more of the listed genes and the composition of gut bacteria in the subject. It is also expected that the bacterial administration will improve behavioral performance in the subject.

### Example 10: Treatment of Social Behavioral Deficit

A human subject who exhibits behaviorally deficit symptoms is identified. The level of corticosterone in the subject is determined. A composition comprising an effective amount of E. faecalis is administered to the subject via oral administration. The administration of E. faecalis is expected to alter the level of a product of a gene of the subject selected from the group consisting of: Crhl (or an ortholog thereof), c-Fos (or an ortholog thereof), Nr3cl (or an ortholog thereof), a human ortholog of any of the listed genes, or a combination of two or more of the listed genes and the composition of gut bacteria in the subject. It is also expected that the bacterial administration will improve behavioral performance in the subject.

In at least some of the previously described embodiments, one or more elements used in an embodiment can interchangeably be used in another embodiment unless such a replacement is not technically feasible. It will be appreciated by those skilled in the art that various other omissions, additions, and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one of skill in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those of skill in the art. The various aspects and embodiments disclosed herein are for purposes of illustration and are not intended to be limiting, with the true scope and spirit being indicated by the following claims.

### REFERENCES CITED

Adams, J. B., Johansen, L. J., Powell, L. D., Quig, D. & Rubin, R. A. Gastrointestinal flora and gastrointestinal status in children with autism--comparisons to typical children and correlation with autism severity. BMC Gastroenterol 11, 22 (2011).

American Psychiatric Association. Diagnostic and statistical manual of mental disorders (5th ed.). (American Psychiatric Publishing, 2013).

Amir, A. et al. Deblur Rapidly Resolves Single-Nucleotide Community Sequence Patterns. mSystems 2 (2017).

Anderson, D. J. Circuit modules linking internal states and social behaviour in flies and mice. Nat Rev Neurosci 17, 692-704 (2016).

Arentsen, T., Raith, H., Qian, Y., Forssberg, H. & Diaz Heijtz, R. Host microbiota modulates development of social preference in mice. Microb Ecol Health Dis 26, 29719 (2015).

Bains, J. S., Wamsteeker Cusulin, J. I. & Inoue, W. Stress-related synaptic plasticity in the hypothalamus. Nat Rev Neurosci 16, 377-388 (2015).

Balamurugan, R., Rajendiran, E., George, S., Samuel, G. V. & Ramakrishna, B. S. Real-time polymerase chain reaction quantification of specific butyrate-producing bacteria, Desulfovibrio and Enterococcus faecalis in the feces of patients with colorectal cancer. J Gastroenterol Hepatol 23, 1298-1303 (2008).

Barlow, J. T., Bogatyrev, S. R. & Ismagilov, R. F. A quantitative sequencing framework for absolute abundance measurements of mucosal and lumenal microbial communities. Nat Commun 11, 2590 (2020).

Bercik, P. et al. The intestinal microbiota affect central levels of brain-derived neurotropic factor and behavior in mice. Gastroenterology 141, 599-609, 609 e591-593 (2011).

Bogatyrev, S. R., Rolando, J. C. & Ismagilov, R. F. Self-reinoculation with fecal flora changes microbiota density and composition leading to an altered bile-acid profile in the mouse small intestine. Microbiome 8, 19 (2020).

Bogatyrev, S. R. & Ismagilov., R. F. Quantitative microbiome profiling in lumenal and tissue samples with broad coverage and dynamic range via a single-step 16S rRNA gene DNA copy quantification and amplicon barcoding. bioRxiv (2020).

Bojovic, K. et al. Gut Microbiota Dysbiosis Associated With Altered Production of Short Chain Fatty Acids in Children With Neurodevelopmental Disorders. Front Cell Infect Microbiol 10, 223 (2020).

Bokulich, N. A. et al. Optimizing taxonomic classification of marker-gene amplicon sequences with QIIME 2's q2-feature-classifier plugin. Microbiome 6, 90 (2018).

Bolyen, E. et al. Reproducible, interactive, scalable and extensible microbiome data science using QIIME 2. Nat Biotechnol 37, 852-857 (2019).

Bolyen, E. et al. Author Correction: Reproducible, interactive, scalable and extensible microbiome data science using QIIME 2. Nat Biotechnol 37, 1091 (2019).

Bravo, J. A. et al. Ingestion of Lactobacillus strain regulates emotional behavior and central GABA receptor expression in a mouse via the vagus nerve. Proc Natl Acad Sci USA 108, 16050-16055 (2011).

Buffington, S. A. et al. Microbial Reconstitution Reverses Maternal Diet-Induced Social and Synaptic Deficits in Offspring. Cell 165, 1762-1775 (2016).

Buie, T. et al. Recommendations for evaluation and treatment of common gastrointestinal problems in children with ASDs. Pediatrics 125 Suppl 1, S19-29 (2010).

Callahan, B. J. et al. DADA2: High-resolution sample inference from Illumina amplicon data. Nat Methods 13, 581-583 (2016).Hoban, A. E. et al. Regulation of prefrontal cortex myelination by the microbiota. Transl Psychiatry 6, e774 (2016).

Chen, P. & Hong, W. Neural Circuit Mechanisms of Social Behavior. Neuron 98, 16-30 (2018).

Clarke, G. et al. The microbiome-gut-brain axis during early life regulates the hippocampal serotonergic system in a sex-dependent manner. Mol Psychiatry 18, 666-673 (2013).

Crumeyrolle-Arias, M. et al. Absence of the gut microbiota enhances anxiety-like behavior and neuroendocrine response to acute stress in rats. Psychoneuroendocrinology 42, 207-217 (2014).

De Palma, G. et al. Microbiota and host determinants of behavioural phenotype in maternally separated mice. Nat Commun 6, 7735 (2015).

De Palma, G. et al. Transplantation of fecal microbiota from patients with irritable bowel syndrome alters gut function and behavior in recipient mice. Sci Transl Med 9 (2017).

Desbonnet, L., Clarke, G., Shanahan, F., Dinan, T. G. & Cryan, J. F. Microbiota is essential for social development in the mouse. Mol Psychiatry 19, 146-148 (2014).

Desbonnet, L. et al. Gut microbiota depletion from early adolescence in mice: Implications for brain and behaviour. Brain Behav lmmun 48, 165-173 (2015).

Diaz Heijtz, R. et al. Normal gut microbiota modulates brain development and behavior. Proc Natl Acad Sci USA 108, 3047-3052 (2011).

Dinan, T. G. & Cryan, J. F. The impact of gut microbiota on brain and behaviour: implications for psychiatry. Curr Opin Clin Nutr Metab Care 18, 552-558 (2015).

Donaldson, Z. R. & Young, L. J. Oxytocin, vasopressin, and the neurogenetics of sociality. Science 322, 900-904 (2008).

Foster, J. A., Rinaman, L. & Cryan, J. F. Stress & the gut-brain axis: Regulation by the microbiome. Neurobiol Stress 7, 124-136 (2017).

Fuzesi, T., Daviu, N., Wamsteeker Cusulin, J. I., Bonin, R. P. & Bains, J. S. Hypothalamic CRH neurons orchestrate complex behaviours after stress. Nat Commun 7, 11937 (2016).

Gacias, M. et al. Microbiota-driven transcriptional changes in prefrontal cortex override genetic differences in social behavior. Elife 5 (2016).

Gareau, M. G. et al. Bacterial infection causes stress-induced memory dysfunction in mice. Gut 60, 307-317 (2011).

Golubeva, A. V. et al. Microbiota-related changes in bile acid and tryptophan metabolism are associated gastrointestinal dysfunction in a mouse model of autism. EbioMedicine, 24, 166-178 (2017).

Gorrindo, P. et al. Gastrointestinal dysfunction in autism: parental report, clinical evaluation, and associated factors. Autism Res 5, 101-108 (2012).

Hagihara, H., Toyama, K., Yamasaki, N. & Miyakawa, T. Dissection of hippocampal dentate gyms from adult mouse. J Vis Exp (2009).

Hanstock, T. L., Clayton, E. H., Li, K. M. & Mallet, P. E. Anxiety and aggression associated with the fermentation of carbohydrates in the hindgut of rats. Physiol Behav 82, 357-368 (2004).

Hoban, A. E. et al. Regulation of prefrontal cortex myelination by the microbiota. Transl Psychiatry 6, e774 (2016).

Hoban, A. E. et al. The microbiome regulates amygdala-dependent fear recall. Mol Psychiatry (2017).

Hsiao, E. Y. et al. Microbiota modulate behavioral and physiological abnormalities associated with neurodevelopmental disorders. Cell 155, 1451-1463 (2013).

Hung, L. W. et al. Gating of social reward by oxytocin in the ventral tegmental area. Science 357, 1406-1411 (2017).

Janssen, S. et al. Phylogenetic Placement of Exact Amplicon Sequences Improves Associations with Clinical Information. mSystems 3 (2018).

Kambe, J. et al. Heat-killed Enterococcus fecalis (EC-12) supplement alters the expression of neurotransmitter receptor genes in the prefrontal cortex and alleviates anxiety-like behavior in mice. Neurosci Lett 720, 134753 (2020).

Kang, D. W. et al. Reduced incidence of Prevotella and other fermenters in intestinal microflora of autistic children. PLoS One 8, e68322 (2013).

Lozupone, C. & Knight, R. UniFrac: a new phylogenetic method for comparing microbial communities. Appl Environ Microbiol 71, 8228-8235 (2005).

Luczynski, P. et al. Adult microbiota-deficient mice have distinct dendritic morphological changes: differential effects in the amygdala and hippocampus. Eur J Neurosci 44, 2654-2666 (2016).

Mayer, E. A., Padua, D. & Tillisch, K. Altered brain-gut axis in autism: comorbidity or causative mechanisms? Bioessays 36, 933-939 (2014).

Mayer, E. A., Knight, R., Mazmanian, S. K., Cryan, J. F. & Tillisch, K. Gut microbes and the brain: paradigm shift in neuroscience. J Neurosci 34, 15490-15496 (2014).

Mazzoli, R. and Pessione, E. The neuro-endocrinological role of microbial glutamate and GABA signaling. Front Microbiol 7, 1934 (2016).

McDonald, D. et al. An improved Greengenes taxonomy with explicit ranks for ecological and evolutionary analyses of bacteria and archaea. ISME J6, 610-618 (2012).

Mohle, L. et al. Ly6C(hi) Monocytes Provide a Link between Antibiotic-Induced Changes in Gut Microbiota and Adult Hippocampal Neurogenesis. Cell Rep 15, 1945-1956 (2016).

Needham, B. D., Tang, W. & Wu, W. L. Searching for the gut microbial contributing factors to social behavior in rodent models. Dev Neurobiol (2018).

Neufeld, K. M., Kang, N., Bienenstock, J. & Foster, J. A. Reduced anxiety-like behavior and central neurochemical change in germ-free mice. Neurogastroenterol Motil 23, 255-264, e119 (2011).

Ogbonnaya, E. S. et al. Adult Hippocampal Neurogenesis Is Regulated by the Microbiome. Biol Psychiatry 78, e7-9 (2015).

Papasian, C. G. et al. Endogenous and exogenous glucocorticoids in experimental enterococcal infection. Clin Vaccine Immunol 13, 349-355 (2006).

Pessione, E. et al. First evidence of a membrane-bound, tyramine and beta-phenylethylamine producing, tyrosine decarboxylase in Enterococcus faecalis: a two-dimensional electrophoresis proteomic study. Proteomics 9, 2695-2710 (2009).

Pressley, L. & Funder, J. W. Glucocorticoid and mineralocorticoid receptors in gut mucosa. Endocrinology 97, 588-596 (1975).

Quast, C. et al. The SILVA ribosomal RNA gene database project: improved data processing and web-based tools. Nucleic Acids Res 41, D590-596 (2013).

Reichardt, S. D. et al. Glucocorticoids enhance intestinal glucose uptake via the dimerized glucocorticoid receptor in enterocytes. Endocrinology 153, 1783-1794 (2012).

Rogers, G. B. et al. From gut dysbiosis to altered brain function and mental illness: mechanisms and pathways. Mol Psychiatry 21, 738-748 (2016).

Rognes, T., Flouri, T., Nichols, B., Quince, C. & Mahe, F. VSEARCH: a versatile open source tool for metagenomics. PeerJ 4, e2584 (2016).

Schroeder, B. O. & Backhed, F. Signals from the gut microbiota to distant organs in physiology and disease. Nat Med 22, 1079-1089 (2016).

Sgritta, M. et al. Mechanisms Underlying Microbial-Mediated Changes in Social Behavior in Mouse Models of Autism Spectrum Disorder. Neuron 101, 246-259 e246 (2019).

Sharon, G., Sampson, T. R., Geschwind, D. H. & Mazmanian, S. K. The Central Nervous System and the Gut Microbiome. Cell 167, 915-932 (2016).

Shih, H. T. & Mok, H. K. ETHOM: Event-recording computer software for the study of animal behavior. Acta Zoologica Taiwanica 11, 47-61 (2000).

Shiina, T. et al. Actions of probiotics on trinitrobenzenesulfonic acid-induced colitis in rats. Biomed Res Int 2015, 528523 (2105).

Shimazu, S. and Miklya, I. Pharmacological studies with endogenous enhancer substances: beta-phenylethylamine, tryptamine, and their synthetic derivatives. Prog Neuropsychopharmacol Biol Psychiatry 28, 421-427 (2004).

Siuda, E. R., Al-Hasani, R., McCall, J. G., Bhatti, D. L. & Bruchas, M. R. Chemogenetic and Optogenetic Activation of Galphas Signaling in the Basolateral Amygdala Induces Acute and Social Anxiety-Like States. Neuropsychopharmacology 41, 2011-2023 (2016).

Spandidos, A., Wang, X., Wang, H. & Seed, B. PrimerBank: a resource of human and mouse PCR primer pairs for gene expression detection and quantification. Nucleic Acids Res 38, D792-799 (2010).

Stilling, R. M. et al. Microbes & neurodevelopment—Absence of microbiota during early life increases activity-related transcriptional pathways in the amygdala. Brain Behav lmmun 50, 209-220 (2015).

Sudo, N. et al. Postnatal microbial colonization programs the hypothalamic-pituitary-adrenal system for stress response in mice. J Physiol 558, 263-275 (2004).

Takahashi, K. et al. Effect of Enterococcus faecalis 2001 on colitis and depressive-like behavior in dextran sulfate sodium-treated mice: involvement of the brain-gut axis. J Neuroinflammation 16, 201 (2019).

Taves, M. D., Gomez-Sanchez, C. E. & Soma, K. K. Extra-adrenal glucocorticoids and mineralocorticoids: evidence for local synthesis, regulation, and function. Am J Physiol Endocrinol Metab 301, E11-24 (2011).

Tillisch, K. et al. Brain Structure and Response to Emotional Stimuli as Related to Gut Microbial Profiles in Healthy Women. Psychosom Med 79, 905-913 (2017).

Urban, D. J. & Roth, B. L. DREADDs (designer receptors exclusively activated by designer drugs): chemogenetic tools with therapeutic utility. Annu Rev Pharmacol Toxicol 55, 399-417 (2015).

Wamsteeker Cusulin, J. I., Fuzesi, T., Watts, A. G. & Bains, J. S. Characterization of corticotropin-releasing hormone neurons in the paraventricular nucleus of the hypothalamus of Crh-IRES-Cre mutant mice. PLoS One 8, e64943 (2013).

Yoshioka, W. et al. Fluorescence laser microdissection reveals a distinct pattern of gene activation in the mouse hippocampal region. Sci Rep 2, 783 (2012).

Zeng, L. et al. Microbiota Modulates Behavior and Protein Kinase C mediated cAMP response element-binding protein Signaling. Sci Rep 6, 29998 (2016).

Zheng, P. et al. Gut microbiome remodeling induces depressive-like behaviors through a pathway mediated by the host's metabolism. Mol Psychiatry 21, 786-796 (2016).

What is claimed is:

1. A method of treating a social behavioral deficit in a subject in need thereof, the method comprising:
identifying a subject having a social behavioral deficit, or a symptom thereof;
administering to the subject a composition comprising an effective amount of one or more Enterococcus bacteria; and
wherein the social behavioral deficit of the subject is improved after administering the composition.

2. The method of claim 1, wherein the social behavioral deficit is anxiety, autism spectrum disorder (ASD), schizophrenia, or depression.

3. The method of claim 1, wherein a sole active ingredient administered to the subject in the method consists essentially of the one or more Enterococcus bacteria.

4. The method of claim 1, wherein the effective amount of the one or more Enterococcus bacteria is in a composition substantially free of bacteria other than the Enterococcus bacteria.

5. The method of claim 1, wherein the one or more Enterococcus bacteria comprises E. faecalis.

6. The method of claim 1, wherein a sole active ingredient administered to the subject in the method consists essentially of E. faecalis.

7. The method of claim 1, wherein the effective amount of the one or more Enterococcus bacteria is in a composition substantially free of bacteria other than the E. faecalis.

8. The method of claim 1, wherein an effective amount of one or more Enterococcus bacteria is administered orally.

9. The method of claim 1, wherein the behavioral performance is determined by standard behavioral testing.

10. The method of claim 1, wherein the composition comprising bacteria within the genus Enterococcus is a probiotic composition.

11. The method of claim 1, wherein the composition comprising bacteria within the genus Enterococcus is a nutraceutical composition.

12. The method of claim 1, wherein the composition comprising bacteria within the genus Enterococcus is a pharmaceutical composition.

13. A method of treating a social behavioral deficit in a subject, the method comprising:
identifying a subject having a social behavioral deficit; and
reducing corticosterone levels in the subject, thereby reducing the social behavioral deficit in the subject.

14. The method of claim 13, wherein reducing corticosterone levels in the subject comprises inhibiting corticosterone synthesis in the subject.

15. The method of claim 13, wherein inhibiting corticosterone synthesis in the subject comprises a pharmacological or a surgical approaches or a combination thereof.

16. The method of claim 15, wherein the pharmacological approach comprises injection of metyrapone in the subject.

17. The method of claim 15, wherein the surgical approach comprises removal of at least one of the two adrenal glands in the subject.

18. The method of claim 14, further comprising determining the corticosterone levels in the subject prior to and after inhibiting corticosterone synthesis in the subject.

19. The method of claim 14, wherein reducing corticosterone levels in the subject comprises:
removing an adrenal gland in the subject;
antagonizing a glucocorticoid receptor in the subject;
inhibiting corticosterone synthesis in the subject; or
promoting gut microbiome levels in the subject,
or a combination thereof.

20. The method of claim 19, wherein removing an adrenal gland comprises removal of at least one of the two adrenal glands.

21. The method of claim 19, further comprising determining the corticosterone levels in the subject prior to and after removing an adrenal gland in the subject.

22. The method of claim 19, wherein antagonizing a glucocorticoid receptor comprises administering RU-486 to the subject.

23. The method of claim 19, further comprising determining the corticosterone levels in the subject prior to and after antagonizing a glucocorticoid receptor in the subject.

24. The method of claim 19, further comprising determining the corticosterone levels in the subject prior to and after inhibiting corticosterone synthesis in the subject.

25. The method of claim 19, wherein promoting gut microbiome levels in the subject comprises administering to the subject a composition comprising an effective amount of one or more Enterococcus bacteria.


SEQUENCE LISTING (4 sequences)

SEQ ID NO: 1 - DNA, 20 bp, Artificial Sequence
  16S Forward
agagtttgatcmtggctcag

SEQ ID NO: 2 - DNA, 19 bp, Artificial Sequence
  16S Reverse
ggttaccttgttacgactt

SEQ ID NO: 3 - DNA, 24 bp, Artificial Sequence
  Specific primer forward
cccttattgttagttgccatcatt

SEQ ID NO: 4 - DNA, 21 bp, Artificial Sequence
  Specific primer reverse
actcgttgtacttcccattgt